SEQUENCE LISTING (82 sequences)

SEQ ID NO: 1 - protein, 59 aa, Human immunodeficiency virus type 1
TLTVQARQLLSGIVQQQNNLLRAIEAQQHLLQLTVWGIKQLQARILAVERYLKDQLLGI

SEQ ID NO: 2 - protein, 38 aa, Artificial
  synthesized
NNLLRAIEAQQHLLQLTVWGIKQLQARILAVERYLKDQ

SEQ ID NO: 3 - protein, 43 aa, Artificial
  synthesized
GSTMGARSMTLTVQARQLLSGIVQQQNNLLRAIEAQQHLLQLT

SEQ ID NO: 4 - protein, 54 aa, Artificial
  synthesized
GSTMGARSMTLTVQARQLLSGIVQQQNNLLRAIEAQQHLLQLTVWGIKQLQARI

SEQ ID NO: 5 - protein, 36 aa, Artificial
  synthesized
GARSMTLTVQARQLLSGIVQQQNNLLRAIEAQQHLL

SEQ ID NO: 6 - protein, 38 aa, Artificial
  synthesized
GARSMTLTVQARQLLSGIVQQQNNLLRAIEAQQHLLQL

SEQ ID NO: 7 - protein, 40 aa, Artificial
  synthesized
GARSMTLTVQARQLLSGIVQQQNNLLRAIEAQQHLLQLTV

SEQ ID NO: 8 - protein, 50 aa, Artificial
  synthesized
GARSMTLTVQARQLLSGIVQQQNNLLRAIEAQQHLLQLTVWGIKQLQARI

SEQ ID NO: 9 - protein, 36 aa, Artificial
  synthesized
ARSMTLTVQARQLLSGIVQQQNNLLRAIEAQQHLLQ

SEQ ID NO: 10 - protein, 36 aa, Artificial
  synthesized
RSMTLTVQARQLLSGIVQQQNNLLRAIEAQQHLLQL

SEQ ID NO: 11 - protein, 36 aa, Artificial
  synthesized
SMTLTVQARQLLSGIVQQQNNLLRAIEAQQHLLQLT

SEQ ID NO: 12 - protein, 35 aa, Artificial
  synthesized
MTLTVQARQLLSGIVQQQNNLLRAIEAQQHLLQLT

SEQ ID NO: 13 - protein, 36 aa, Artificial
  synthesized
MTLTVQARQLLSGIVQQQNNLLRAIEAQQHLLQLTV

SEQ ID NO: 14 - protein, 34 aa, Artificial
  synthesized
TLTVQARQLLSGIVQQQNNLLRAIEAQQHLLQLT

SEQ ID NO: 15 - protein, 35 aa, Artificial
  synthesized
TLTVQARQLLSGIVQQQNNLLRAIEAQQHLLQLTV

SEQ ID NO: 16 - protein, 36 aa, Artificial
  synthesized
TLTVQARQLLSGIVQQQNNLLRAIEAQQHLLQLTVW

SEQ ID NO: 17 - protein, 37 aa, Artificial
  synthesized
TLTVQARQLLSGIVQQQNNLLRAIEAQQHLLQLTVWG

SEQ ID NO: 18 - protein, 38 aa, Artificial
  synthesized
TLTVQARQLLSGIVQQQNNLLRAIEAQQHLLQLTVWGI

SEQ ID NO: 19 - protein, 44 aa, Artificial
  synthesized
TLTVQARQLLSGIVQQQNNLLRAIEAQQHLLQLTVWGIKQLQAR

SEQ ID NO: 20 - protein, 36 aa, Artificial
  synthesized
LTVQARQLLSGIVQQQNNLLRAIEAQQHLLQLTVWG

SEQ ID NO: 21 - protein, 42 aa, Artificial
  synthesized
QARQLLSGIVQQQNNLLRAIEAQQHLLQLTVWGIKQLQARIL

SEQ ID NO: 22 - protein, 47 aa, Artificial
  synthesized
QARQLLSGIVQQQNNLLRAIEAQQHLLQLTVWGIKQLQARILAVERY

SEQ ID NO: 23 - protein, 49 aa, Artificial
  synthesized
QARQLLSGIVQQQNNLLRAIEAQQHLLQLTVWGIKQLQARILAVERYLK

SEQ ID NO: 24 - protein, 51 aa, Artificial
  synthesized
QARQLLSGIVQQQNNLLRAIEAQQHLLQLTVWGIKQLQARILAVERYLKDQ

SEQ ID NO: 25 - protein, 36 aa, Artificial
  synthesized
SGIVQQQNNLLRAIEAQQHLLQLTVWGIKQLQARIL

SEQ ID NO: 26 - protein, 45 aa, Artificial
  synthesized
SGIVQQQNNLLRAIEAQQHLLQLTVWGIKQLQARILAVERYLKDQ

SEQ ID NO: 27 - protein, 41 aa, Artificial
  synthesized
QQQNNLLRAIEAQQHLLQLTVWGIKQLQARILAVERYLKDQ

SEQ ID NO: 28 - protein, 34 aa, Artificial
  synthesized
RAIEAQQHLLQLTVWGIKQLQARILAVERYLKDQ

SEQ ID NO: 29 - protein, 41 aa, Artificial
  synthesized
QQQNNLLRAIEAQQHLLQLTAWGIKQLQARILAVERYLKDQ

SEQ ID NO: 30 - protein, 41 aa, Artificial
  synthesized
QQQNNLLRAIEAQQHLLQLTVAGIKQLQARILAVERYLKDQ

SEQ ID NO: 31 - protein, 49 aa, Artificial
  synthesized
QARQLLSGIVQQQNNLLRAIEAQQHLLQLTVFGIRQLQARILAVERYLK

SEQ ID NO: 32 - protein, 49 aa, Artificial
  synthesized
QARQLLSGIVQQQNNLLRAIEAQQHALQATVWGIKQLQARILAVERYLK

SEQ ID NO: 33 - protein, 36 aa, Artificial
  synthesized
WMEWDREINNYTSLIHSLIEESQNQQEKNEQELLEL

SEQ ID NO: 34 - protein, 51 aa, Artificial
  synthesized
QARQLLSGIVQQQNNLLRAIEAQQHALQATVWGIKQLQARILAVERYLKDQ

SEQ ID NO: 35 - protein, 49 aa, Artificial
  synthesized
QARQLVSGLVQQQNNILRALEATQHAVQALVWGVKQLQARVLALERYIK

SEQ ID NO: 36 - protein, 49 aa, Artificial
  synthesized
QIRQLLSGIVQQQNNLLRAIEAIQHALQAIVWGIKQLQARILAVERYLK

SEQ ID NO: 37 - protein, 49 aa, Artificial
  synthesized
QARQLVSGLVQQQNNILRALEATQHAVQALVWGVRQLQARVLALERYIK

SEQ ID NO: 38 - protein, 51 aa, Artificial
  synthesized
QARQLLSGIVQQQNNLLRAIEATQHAVQALVWGVKQLQARVLALERYIKDQ

SEQ ID NO: 39 - protein, 51 aa, Artificial
  synthesized
QARQLVSGLVQQQNNILRALEAQQHALQATVWGIKQLQARVLALERYIKDQ

SEQ ID NO: 40 - protein, 51 aa, Artificial
  synthesized
QARQLLSGIVQQQNNLLRAIEAQQHALQATVWGVKQLQARILAVERYLKDQ

SEQ ID NO: 41 - protein, 49 aa, Artificial
  synthesized
QARQLVSGLVQQQNNILRALEATQHLVQLLVWGVKQLQARVLALERYIK

SEQ ID NO: 42 - protein, 49 aa, Artificial
  synthesized
QIRQLLSGIVQQQNNLLRAIEAIQHLLQLIVWGIKQLQARILAVERYLK

SEQ ID NO: 43 - protein, 41 aa, Artificial
  synthesized
QQQNNLLRAIEAQQHLLQLTVFGIKQLQARILAVERYLKDQ

SEQ ID NO: 44 - protein, 41 aa, Artificial
  synthesized
QQQNNLLRAIEAQQHLLQLTVWGIAQLQARILAVERYLKDQ

SEQ ID NO: 45 - protein, 41 aa, Artificial
  synthesized
QQQNNLLRAIEAQQHLLQLTVWGIKQLAARILAVERYLKDQ

SEQ ID NO: 46 - protein, 41 aa, Artificial
  synthesized
QQQNNLLRAIEAQQHALQLTVWGIKQLQARILAVERYLKDQ

SEQ ID NO: 47 - DNA, 147 bp, Artificial
  synthesized
caggctcgtcagctgctgtctggtatcgttcagcagcagaacaacctgctgcgtgctatcgaagctcagcagcacgctctgcaggctaccgtttggggtatcaaacagctgcaggctcgtatcctggctgttgaacgttacctgaaa

SEQ ID NO: 48 - DNA, 147 bp, Artificial
  synthesized
caggctcgtcagctggtttctggtctggttcagcagcagaacaacatcctgcgtgctctggaagctacccagcacgctgttcaggctctggtttggggtgttaaacagctgcaggctcgtgttctggctctggaacgttacatcaaa

SEQ ID NO: 49 - DNA, 147 bp, Artificial
  synthesized
cagatccgtcagctgctgtctggtatcgttcagcagcagaacaacctgctgcgtgctatcgaagctatccagcacgctctgcaggctatcgtttggggtatcaaacagctgcaggctcgtatcctggctgttgaacgttacctgaaa

SEQ ID NO: 50 - DNA, 123 bp, Artificial
  synthesized
cagcagcagaacaacctgctgcgtgctatcgaagctcagcagcacctgctgcagctgaccgcttggggtatcaaacagctgcaggctcgtatcctggctgttgaacgttacctgaaagaccag

SEQ ID NO: 51 - DNA, 123 bp, Artificial
  synthesized
cagcagcagaacaacctgctgcgtgctatcgaagctcagcagcacctgctgcagctgaccgttgctggtatcaaacagctgcaggctcgtatcctggctgttgaacgttacctgaaagaccag

SEQ ID NO: 52 - DNA, 147 bp, Artificial
  synthesized
caggcccgccagctgctgtccggcatcgtgcagcagcagaacaacctgctgcgcgccatcgaggcccagcagcacgccctgcaggccaccgtgtggggcatcaagcagctgcaggcccgcatcctggccgtggagcgctacctgaag

SEQ ID NO: 53 - DNA, 147 bp, Artificial
  synthesized
caggcccgccagctggtgtccggccgcgtgcagcagcagaacaacatcctgcgcgccctggaggccacccagcacgccgtgcaggccctggtgtggggcgtgaagcagctgcaggcccgcgtgctggccctggagcgctacatcaag

SEQ ID NO: 54 - DNA, 147 bp, Artificial
  synthesized
cagatccgccagctgctgtccggcatcgtgcagcagcagaacaacctgctgcgcgccatcgaggccatccagcacgccctgcaggccatcgtgtggggcatcaagcagctgcaggcccgcatcctggccgtggagcgctacctgaag

SEQ ID NO: 55 - DNA, 123 bp, Artificial
  synthesized
cagcagcagaacaacctgctgcgcgccatcgaggcccagcagcacctgctgcagctgaccgcctggggcatcaagcagctgcaggcccgcatcctggccgtggagcgctacctgaaggaccag

SEQ ID NO: 56 - DNA, 123 bp, Artificial
  synthesized
cagcagcagaacaacctgctgcgcgccatcgaggcccagcagcacctgctgcagctgaccgtggccggcatcaagcagctgcaggcccgcatcctggccgtggagcgctacctgaaggaccag

SEQ ID NO: 57 - protein, 8 aa, Artificial
  synthesized; Xaa is any amino acid
WXXWXXXI

SEQ ID NO: 58 - protein, 7 aa, Artificial
  synthesized; Xaa is any amino acid
WXXWXXX

SEQ ID NO: 59 - protein, 6 aa, Artificial
  synthesized; Xaa is any amino acid
WXXWXX

SEQ ID NO: 60 - protein, 5 aa, Artificial
  synthesized; Xaa is any amino acid
WXXWX

SEQ ID NO: 61 - protein, 4 aa, Artificial
  synthesized; Xaa is any amino acid
WXXW

SEQ ID NO: 62 - protein, 8 aa, Artificial
  synthesized; Xaa is any amino acid
WXXXWXWX

SEQ ID NO: 63 - protein, 7 aa, Artificial
  synthesized; Xaa is any amino acid
XXXWXWX

SEQ ID NO: 64 - protein, 6 aa, Artificial
  synthesized; Xaa is any amino acid
XXWXWX

SEQ ID NO: 65 - protein, 5 aa, Artificial
  synthesized; Xaa is any amino acid
XWXWX

SEQ ID NO: 66 - protein, 4 aa, Artificial
  synthesized; Xaa is any amino acid
WXWX

SEQ ID NO: 67 - protein, 7 aa, Artificial
  synthesized; Xaa is any amino acid
WXXXWXW

SEQ ID NO: 68 - protein, 6 aa, Artificial
  synthesized; Xaa is any amino acid
WXXXWX

SEQ ID NO: 69 - protein, 5 aa, Artificial
  synthesized; Xaa is any amino acid
WXXXW

SEQ ID NO: 70 - protein, 8 aa, Artificial
  synthesized; Xaa is any amino acid
IXXXWXXW

SEQ ID NO: 71 - protein, 7 aa, Artificial
  synthesized; Xaa is any amino acid
XXXWXXW

SEQ ID NO: 72 - protein, 6 aa, Artificial
  synthesized; Xaa is any amino acid
XXWXXW

SEQ ID NO: 73 - protein, 5 aa, Artificial
  synthesized; Xaa is any amino acid
XWXXW

SEQ ID NO: 74 - protein, 8 aa, Artificial
  synthesized; X is any amino acid
XWXWXXXW

SEQ ID NO: 75 - protein, 7 aa, Artificial
  synthesized; Xaa is any amino acid
XWXWXXX

SEQ ID NO: 76 - protein, 6 aa, Artificial
  synthesized; Xaa is any amino acid
XWXWXX

SEQ ID NO: 77 - protein, 4 aa, Artificial
  synthesized; Xaa is any amino acid
XWXW

SEQ ID NO: 78 - protein, 7 aa, Artificial
  synthesized; Xaa is any amino acid
WXWXXXW

SEQ ID NO: 79 - protein, 6 aa, Artificial
  synthesized; Xaa is any amino acid
XWXXXW

SEQ ID NO: 80 - protein, 7 aa, Artificial
  synthesized; Xaa is any amino acid
WXXXWXX

SEQ ID NO: 81 - protein, 51 aa, Artificial
  synthesized; X is any amino acid
QARQLLSGIVQQQNNLLRAIEAQQHLLQATVWGIKQLQARILAVERYLKDQ

SEQ ID NO: 82 - protein, 41 aa, Artificial
  synthesized; X is any amino acid
QQQNNLLRAIEAQQHLLQATVWGIKQLQARILAVERYLKDQ

(19) United States
(12) Patent Application Publication
(10) Pub. No.: US 2004/0076637 A1
(43) Pub. Date: Apr. 22, 2004

(54) HIV-derived HR1 peptides modified to form stable trimers, and their use in therapy to inhibit transmission of human immunodeficiency virus

(76) Inventors: Mary Kay Delmedico, Raleigh, NC (US); John Dwyer, Chapel Hill, NC (US)

Correspondence Address: M. Bud Nelson, Trimeris, Inc., 3518 Westgate Dr., Suite 300, Durham, NC, 27707, US

(21) Appl. No.: 10/664,021

(22) Filed: Sep. 16, 2003

Related U.S. Application Data
(60) Provisional application No. 60/414,514, filed on Sep. 27, 2002.

(51) Int. Cl. A61K 39/21; C07K 14/16

(52) U.S. Cl. 424/188.1; 530/350

(57) ABSTRACT

Provided are synthetic peptides comprising amino acid sequence from the HR1 region of HIV-1 gp41, and having one or more amino acid substitutions in a hydrophobic domain of the HR1 region of HIV-1 gp41. As a result of the one or more amino acid substitutions in the hydrophobic domain, synthetic peptide self-assembles into trimers in solution. Also provided are trimers resulting from self-association of synthetic peptide. Additionally provided are methods of using these compositions to inhibit transmission of HIV to a target cell, or to inhibit HIV fusion to a target cell, wherein the methods involve adding an effective amount of synthetic peptide (or trimer formed therefrom) to inhibit viral transmission or viral fusion, respectively.

This application claims the benefit of the U.S. Provisional Application 60/414,514 filed on 27 Sep. 2002.

### FIELD OF THE INVENTION

The present invention relates to synthetic peptides derived from the HR1 region of gp41, trimers formed therefrom, and their uses in anti-HIV therapy as antiviral agents to inhibit transmission of HIV (Human Immunodeficiency Virus) to target cells. More particularly, the present invention comprises a family of synthetic peptides which contain one or more site-specific amino acid substitutions (as compared to the native sequence) which uniquely result in a change in the oligomerization state in solution to self-association into predominately a trimeric form in forming a coiled coil, and may further stabilize such trimers formed.

### BACKGROUND OF THE INVENTION

It is now well known that cells can be infected by HIV through a process by which fusion occurs between the cellular membrane and the viral membrane. The generally accepted model of this process is that the viral envelope glycoprotein complex (gp120/gp41) interacts with cell surface receptors on the membranes of the target cells. Following binding of gp120 to cellular receptors (e.g., CD4 in combination with a chemokine co-receptor such as CCR-5 or CXCR-4), induced is a conformational change in the gp120/gp41 complex that allows gp41 to insert into the membrane of the target cell and mediate membrane fusion.

The amino acid sequence of gp41, and its variation among different strains of HIV, is well known. FIG. 1 is a schematic representation of the generally accepted functional domains of gp41 (note the amino acid sequence numbers may vary slightly depending on the HIV-1 strain). The fusion peptide (fusogenic domain) is believed to be involved in insertion into and disruption of the target cell membrane. The transmembrane domain, containing the transmembrane anchor sequence, is located at the C-terminal end of the protein. Between the fusion peptide and transmembrane anchor are two distinct regions, known as heptad repeat (HR) regions, each region having a plurality of heptads. The HR1 region, nearer to the N-terminal end of the protein than the HR2 region as depicted in FIG. 1, has been generally described as comprising an amino acid sequence having the sequence of SEQ ID NO:1. However, due to naturally occurring polymorphisms, the amino acid sequence of the HR1 region of HIV-1 gp41 may vary slightly, depending on the viral strain from which the amino acid sequence was deduced. The amino acid sequence comprising the HR1 region is one of the most highly conserved regions in the HIV-1 envelope protein (Shu et al., 1999, Biochemistry, 38:5378-5385). The other region, HR2, is also depicted in FIG. 1 wherein the amino acid numbering corresponds to the amino acid sequence of gp160 in strain III B. The HR regions are known to have a plurality of 7 amino acid residue stretches or “heptads” (the 7 amino acids in each heptad designated “a” through “g”), wherein the amino acids in the “a” position and “d” position are generally hydrophobic. Also present in each HR region is one or more leucine zipper-like motifs (also referred to as “leucine zipper-like repeats”), each comprising an 8 amino acid sequence initiating with, and ending with an isoleucine or leucine. Most frequently, the HR2 region has just one leucine zipper like-motif, whereas the HR1 region has five leucine zipper-like motifs. Heptad repeats and leucine zipper-like motifs are amino acid sequences that contribute to formation of a coiled coil structure of gp41, and of a coiled coil structure of peptides derived from the HR regions. Generally, coiled coils are known to be comprised of two or more helices that wrap around each other in forming oligomers, with the hallmark of coiled coils being a heptad repeat of amino acids with a predominance of hydrophobic residues at the first (“a”) and fourth (“d”) positions, charged residues frequently at the fifth (“e”) and seventh (“g”) positions, and with the amino acids in the “a” position and “d” position being primary determinants that influence the oligomeric state and strand orientation (see, e.g., Akey et al., 2001, Biochemistry, 40:6352-60).

It was discovered that peptides derived from either the HR1 region (“HR1 peptides”) or HR2 region (“HR2 peptides”) of HIV-1 gp41 inhibit transmission of HIV to host cells both in in vitro assays and in in vivo clinical studies (see, e.g., Wild et al., 1994, Proc. Natl. Acad. Sci. USA, 91:9770-9774; U.S. Pat. Nos. 5,464,933 and 5,656,480 licensed to the present assignee; and Kilby et al., 1998, Nature Med. 4:1302-1306. See also, e.g., U.S. Pat. Nos. 6,258,782 and 6,348,568 assigned to the present assignee. The disclosures of these patents are herein incorporated by reference). More particularly, HR1 peptides exemplified by DP107 (also known as T-21, a synthetic peptide having the amino acid sequence of SEQ ID NO:2) blocked infection of T cells with 50% effective concentration values (EC50) of 1 μg/ml (see, e.g., Lawless et al., 1996, Biochemistry, 35:13697-13708). Sedimentation equilibrium experiments indicated that, in solution, T-21 peptide exists in a monomer/dimer/tetramer equilibrium (e.g., at concentrations of 5 μM or less, with predominately tetramers at high concentrations of peptide (e.g., 10 μM or more). A structural interaction occurring between a HR2 peptide and HR1 peptide has been observed when HR1 peptide is tetrameric (Lawless et al., supra). However, the generally accepted model of gp41 suggests that the gp41 core exists as a six helix bundle comprised of three N-terminal (HR1) regions forming a parallel trimeric coiled coil, where three C-terminal (HR2) regions pack in an antiparallel orientation into the hydrophobic grooves on the surface of the trimeric coiled coil (see, e.g., Shu et al., 1999, Biochemistry 38:5378-5385; Root et al., 2001, Science 291:884-888, and U.S. Pat. No. 6,150,088). Accordingly, as compared to monomeric or tetrameric structures, trimers formed from self-assembly of synthetic peptide may provide a structure that acts more like the trimeric HR1 region in the in vivo binding interactions between the trimeric HR1 region and trimeric HR2 region of HIV gp41.

Thus, there is a need for additional compounds (particularly synthetic peptides self-assembled into trimeric form) which can interfere with the interaction of the various domains of gp41 involved in oligomerization and with the changes that gp41 undergoes which are necessary to effect fusion, thereby inhibiting the fusion of HIV gp41 to a target cell membrane.

### SUMMARY OF THE INVENTION

The present invention relates to synthetic peptides derived from the HR1 region of HIV-1 gp41 wherein the synthetic peptides contain one or more site-specific amino acid substitutions (as compared to the native sequence of that HR1 region of HIV-1 gp41 or HR1 peptide derived therefrom), in one or more of the plurality of heptads of the peptide, which unexpectedly result in a change in the oligomerization state in solution to self-association into predominately a trimeric form (“self-associates” or “self-assembles” “into trimers”). Also provided are trimers formed from synthetic peptide.

In another object of the invention, provided are synthetic peptides derived from the HR1 region of HIV-1 gp41 which, in addition to containing one or more site-specific amino acid substitutions in one or more of the plurality of heptads which result in self-assembly into a trimeric form, also contains a plurality of amino acid substitutions which unexpectedly stabilize such trimers formed. Also provided are trimers formed from synthetic peptide.

In another object of the present invention, provided are synthetic peptides derived from the HR1 region of HIV-1 gp41 wherein the peptides (a) contain one or more site-specific amino acid substitutions (as compared to the native sequence of the HR1 region of HIV-1 gp41), in one or more of the plurality of heptads of such peptide, which unexpectedly result in self-association into predominantly a trimeric form in solution; and

(b) possess antiviral activity as evidenced by their ability to inhibit transmission of HIV to a target cell. Also provided are trimers formed from synthetic peptide.

In another object of the present invention, provided are synthetic peptides derived from the HR1 region of HIV-1 gp41 wherein the peptides (a) contain one or more site-specific amino acid substitutions (as compared to the native sequence of that HR1 region of HIV-1 gp41) in one or more of the plurality of heptads which unexpectedly result in self-association into a trimeric form in solution; (b) contain a plurality of substitutions in the heptad repeat region which unexpectedly stabilize such trimers formed; and (c) possess (preferably when in trimeric form) antiviral activity as evidenced by their ability to inhibit transmission of HIV to a target cell. Also provided are trimers formed from synthetic peptide.

In another object of the present invention, provided is a trimer comprised of synthetic peptide derived from the HR1 region of HIV-1 gp41 wherein the synthetic peptide is comprised of a plurality of heptads (preferably contiguous so as to form a heptad repeat), and wherein the synthetic peptide contains one or more site-specific amino acid substitutions (as compared to the native sequence of that HR1 region of HIV1 gp41 or HR1 peptide derived therefrom) in one or more of the plurality of heptads which unexpectedly results in self-association into predominately a trimeric form in solution. In a preferred embodiment, the trimer comprises a coiled coil.

Also, according to the present invention, provided is a method for inhibition of transmission of HIV-1 to a target cell, comprising contacting the virus, in the presence of the cell, with synthetic peptide according to the present invention (preferably in its self-assembled, trimeric form) in a concentration effective to inhibit infection of the cell by HIV-1. A method for inhibition of transmission of HIV to a target cell also comprises adding synthetic peptide according to the present invention (preferably in its self-assembled, trimeric form) to the virus and the cell in an amount effective to inhibit infection of the cell by HIV. Also provided is a method for inhibiting HIV (preferably, HIV-1) fusion (e.g., a process by which HIV gp41 mediates fusion between the viral membrane and cell membrane during infection by HIV of a target cell), comprising contacting the virus, in the presence of a target cell, with synthetic peptide according to the present invention (preferably in its self-assembled, trimeric form) in a concentration effective to inhibit HIV membrane fusion. These methods may be used to treat HIV-infected individuals. The above and other objects, features, and advantages of the present invention will be apparent in the following Detailed Description of the Invention when read in conjunction with accompanying drawings.

### BRIEF DESCRIPTION OF THE DRAWINGS

FIG. 1 is a schematic of HIV-1 gp41 showing the heptad repeat 1 region (HR1; SEQ ID NO:1) and heptad repeat 2 region (HR2) along with other functional regions of gp41. Exemplary amino acid sequences corresponding to HIV_{IIIB} HR1 and HR2 are shown for purposes of illustration. The amino acid residues are numbered according to their position in gp160, strain HIV_{IIIB}.

FIG. 2 shows a comparison of the sequences contained within the HR1 region of HIV-1 gp41 for purposes of illustration, and not limitation, as determined from various laboratory strains and clinical isolates, wherein illustrated are some of the variations in amino acid sequence, as indicated by the single letter amino acid code.

FIG. 3 shows a comparison between native sequence and illustrative synthetic peptides according to the present invention, wherein substitutions are underlined.

FIG. 4 is a graph showing a comparison of binding curves between HR2 peptide (having the amino acid sequence of SEQ ID NO:33) with native HR1 peptides having the amino acid sequences of SEQ ID NOs:24 or 23, or synthetic peptides having the amino acid sequences of SEQ ID NOs:32 or 36.

### DETAILED DESCRIPTION OF THE INVENTION

Definitions:

The term “individual”, when used herein for purposes of the specification and claims, means a mammal, and preferably a human.

The term “target cell”, when used herein for purposes of the specification and claims, means a cell capable of being infected by HIV. Preferably, the cell is a human cell or are human cells; and more preferably, human cells capable of being infected by HIV-1 via a process including membrane fusion.

The term “pharmaceutically acceptable carrier”, when used herein for purposes of the specification and claims, means a carrier medium that does not significantly alter the biological activity of the active ingredient (e.g., a synthetic peptide or trimer according to the present invention, or a compound discovered according to a method of the present invention) to which it is added. As known to those skilled in the art, a suitable pharmaceutically acceptable carrier may comprise one or substances, including but not limited to, water, buffered water, saline, 0.3% glycine, aqueous alcohols, isotonic aqueous buffer; and may further include one or more substances such as water-soluble polymer, glycerol, polyethylene glycol, glycerin, oils, salts such as sodium, potassium, magnesium and ammonium, phosphonates, carbonate esters, fatty acids, saccharides, polysaccharides, glycoproteins (for enhanced stability), excipients, and preservatives and/or stabilizers (to increase shelf-life or as necessary and suitable for manufacture and distribution of the composition). Preferably, the carrier is suitable for intravenous, intramuscular, subcutaneous or parenteral administration (e.g., by injection).

By the term “amino acid” is meant, for purposes of the specification and claims and in reference to the synthetic peptides according to the present invention, to refer to a molecule that has at least one free amine group and at least one free carboxyl group. The amino acid may have more than one free amine group, or more than one free carboxyl group, or may further comprise one or more free chemical reactive groups other than an amine or a carboxyl group (e.g., a hydroxyl, a sulfhydryl, etc.). The amino acid may be a naturally occurring amino acid (e.g., L-amino acid), a non-naturally occurring amino acid (e.g., D-amino acid), a synthetic amino acid, a modified amino acid, an amino acid derivative, an amino acid precursor, and a conservative substitution. One skilled in the art would know that the choice of amino acids incorporated into a peptide will depend, in part, on the specific physical, chemical or biological characteristics required of the antiviral peptide. Such characteristics are determined, in part, by determination of trimeric structure (as described herein in more detail) and antiviral activity (as described herein in more detail). For example, the skilled artisan would know from the descriptions herein that amino acids in a synthetic peptide may be comprised of one or more of naturally occurring (L)-amino acid and non-naturally occurring (D)-amino acid. A preferred amino acid may be used with the present invention to the exclusion of amino acids other than the preferred amino acid.

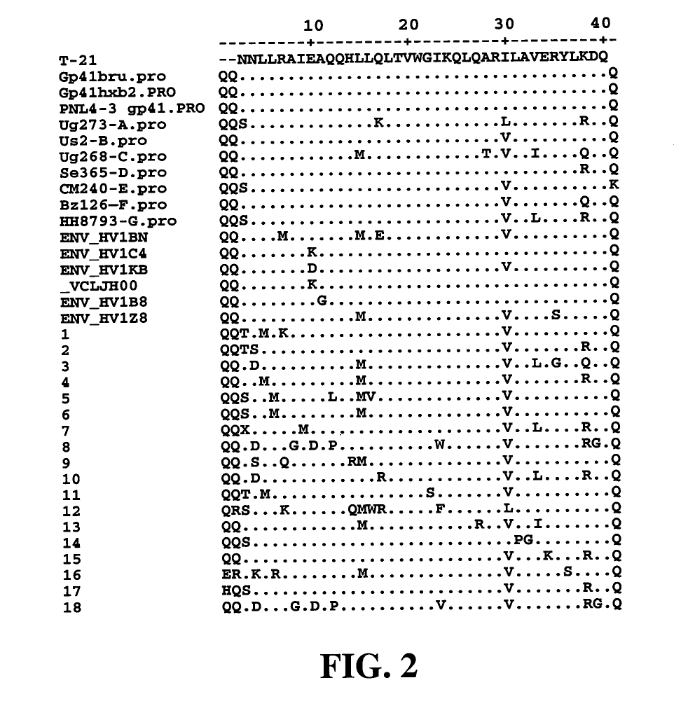

The term “amino acid substitution” is used in relation to the amino acid sequence of a native sequence of the HR1 region of HIV-1 gp41, and is also used in relation to amino acid sequence of a synthetic peptide according to the present invention. The term “amino acid substitution” is used in relation to the native sequence, hereinafter for the purposes of the specification and claims, to mean one or more amino acids substitution in or to the amino sequence of the native sequence in producing a synthetic peptide that can self-assemble in solution into a trimeric form, and wherein the synthetic peptide can bind the HR2 region or a peptide derived thereform, as may be determined by the teachings herein and by using methods routine in the art. Likewise, when comparing synthetic peptides according to the present invention, reference is often made to the amino acid sequence of one synthetic peptide as containing one or more additional amino acid substitutions when compared to the amino acid sequence of another synthetic peptide. Preferably, an amino acid substitution in the amino acid sequence of the synthetic peptide according to the present invention (e.g., as compared to that of a native sequence) ranges from 1 to 10 amino acids, and more preferably from 1 to 5 amino acids (the higher range being more desirable for longer peptides, e.g., about 40 or more amino acids in length). The amino acid substitution may comprise a “conservative substitution”. As known in the art “conservative substitution” is defined by aforementioned function, and includes substitutions of amino acids having substantially the same charge, size, hydrophilicity, and/or aromaticity as the amino acid replaced. Such conservative substitutions are known to those of ordinary skill in the art to include, but are not limited to, glycine-alanine-valine; isoleucine-leucine; tryptophan-tyrosine; aspartic acid-glutamic acid; arginine-lysine; asparagine-glutamine; and serine-threonine. Such substitutions may also comprise one or more of the polymorphisms, as illustrated in FIG. 2, at the various amino acid positions along the HR1 region found in laboratory and/or clinical isolates of HIV-1.

The term “native sequence”, when used herein for purposes of the specification and claims and in reference to the amino acid sequence of the HR1 region of HIV gp41, means a naturally occurring sequence found in laboratory HIV strains and/or HIV clinical isolates. Such sequences are readily available from public gene databases such as GenBank. For purposes of illustration, but not limitation, some of such native sequences are illustrated in FIG. 2, in which illustrative substitutions (e.g., polymorphisms) are noted in various amino acid positions in the amino acid sequence of that portion of the HR1 region of HIV-1 gp41 illustrated. More particularly, a “native sequence” means the amino acid sequence derived from the HR1 region of HIV gp41, which is identical to the amino acid sequence of the synthetic peptide to which the native sequence is compared, except that the native sequence lacks the one or more amino acid substitutions contained in the synthetic peptide which confer the ability of the synthetic peptide to self-assemble in solution into trimers. For example, SEQ ID NO:23 comprises the amino acid sequence of a native sequence when compared to SEQ ID NO:32 which comprises the amino acid sequence of a synthetic peptide of the present invention.

The term “reactive functionality”, when used herein for purposes of the specification and claims, means a chemical group or chemical moiety that is capable of forming a covalent bond and/or is protective (e.g., protects peptide derivatives from reacting with themselves). With respect to chemical groups, a reactive functionality is known to those skilled in the art to comprise a group that includes, but is not limited to, maleimide, thiol, carboxy, phosphoryl, acyl, hydroxyl, acetyl, hydrophobic, amido, dansyl, fluorenylmethyoxycarbonyl (Fmoc), t-butyloxycarbonyl (Boc), sulfo, a succinimide, a thiol-reactive, an amino-reactive, a carboxyl-reactive, and the like. A chemical moiety may comprise a linker. Linkers are known to refer to a compound or moeity that acts as a molecular bridge to operably link two different molecules (e.g., a wherein one portion of the linker binds to a peptide according to the present invention, and wherein another portion of the linker binds to a macromolecular carrier or another antiviral peptide known to inhibit HIV transmission to a target cell). The two different molecules may be linked to the linker in a step-wise manner. There is no particular size or content limitations for the linker so long as it can fulfill its purpose as a molecular bridge. Linkers are known to those skilled in the art to include, but are not limited to, chemical chains, chemical compounds (e.g., reagents), and the like. The linkers may include, but are not limited to, homobifunctional linkers and heterobifunctional linkers. Heterobifunctional linkers, well known to those skilled in the art, contain one end having a first reactive functionality to specifically link a first molecule, and an opposite end having a second reactive functionality to specifically link to a second molecule. It will be evident to those skilled in the art that a variety of bifunctional or polyfunctional reagents, both homo- and heterofunctional (such as those described in the catalog of the Pierce Chemical Co., Rockford, Ill.), may be employed as a linker with respect to the present invention. Depending on such factors as the molecules to be linked, and the conditions in which the linking is performed, the linker may vary in length and composition for optimizing such properties as preservation of biological function stability, resistance to certain chemical and/or temperature parameters, and of sufficient stereo-selectivity or size. For example, the linker should not significantly interfere with the ability of the synthetic peptide according to the present invention (to which it is linked) to function as an inhibitor of either or both of HIV fusion and HIV transmission to a target cell. A preferred reactive functionality may be used with the present invention to the exclusion of a reactive functionality other than the preferred reactive functionality.

The term “macromolecular carrier”, when used herein for purposes of the specification and claims, means a molecule which is linked, joined, or fused (e.g., chemically or through recombinant means) to one or more synthetic peptides according to the present invention, whereby the molecule is capable of conferring one or more of stability to the one or more peptides, increase in biological activity of the one or more synthetic peptides, or increase in serum half-life of the one or more synthetic peptides (e.g., prolonging the persistence of the one or more synthetic peptides in the body) relative to that with respect to the one or more synthetic peptides in the absence of the molecule. Such macromolecular carriers are well known in the art to include, but are not limited to, serum proteins, polymers, carbohydrates, fatty acids, lipids, and lipid-fatty acid conjugates. Serum proteins typically used as macromolecular carriers include, but are not limited to, transferrin, albumin (preferably human), immunoglobulins (preferably human IgG or one or more chains thereof), or hormones. Polymers typically used as macromolecular carriers include, but are not limited to, polylysines or poly(D-L-alanine)-poly(L-lysine)s, or polyols. A preferred polyol comprises a water-soluble poly(alkylene oxide) polymer, and can have a linear or branched chain. Suitable polyols include, but are not limited to, polyethylene glycol (PEG), polypropylene glycol (PPG), and PEG-PPG copolymers. A preferred polyol comprises PEG having an average molecular size selected from the range of from about 1,000 Daltons to about 20,000 Daltons. Other types of macromolecular carriers that can be used, which generally have molecular weights higher than 20,000, are known in the art. A preferred macromolecular carrier may be used with the present invention to the exclusion of a macromolecular carrier other than the preferred macromolecular carrier.

The term “synthetic”, in relation to a peptide according to the present invention, is used hereinafter for the purposes of the specification and claims to mean that the peptide is produced by chemical synthesis, recombinant expression, biochemical or enzymatic fragmentation of a larger molecule, chemical cleavage of larger molecule, a combination of the foregoing or, in general, made by any other method in the art, and isolated. The term “isolated” when used in reference to a synthetic peptide, means that the synthetic peptide is substantially free of components which have not become part of the integral structure of the synthetic peptide itself; e.g., such as substantially free of cellular material or culture medium when produced by recombinant techniques, or substantially free of chemical precursors or other chemicals when chemically synthesized or produced using biochemical or chemical processes. With reference to a “synthetic peptide” which “self-assembles into trimers” (or a “trimeric form”) is meant that when multiple molecules of synthetic peptide are present in a solution (e.g., an aqueous solution, or physiological solution, or pharmaceutically acceptable carrier) such molecules of synthetic peptide can self-assemble into trimers, wherein a trimer is comprised of three molecules of synthetic peptide, and wherein trimers are the predominant (preferred) oligomeric form of the synthetic peptide (with aminority, if detectable at all, of oligomeric forms other than a trimer) present in the solution, as can be determined using standard methods known in the art.

As previously summarized herein, the hallmark of coiled coils is that they comprise a heptad repeat of amino acids with each heptad having a predominance of hydrophobic residues at the first (“a”) and fourth (“d”) positions, charged residues frequently at the fifth (“e”) and seventh (“g”) positions, and with the amino acids in the “a” position and “d” position being determinants that influence the oligomeric state and strand orientation (see, e.g., Akey et al., 2001, Biochemistry, 40:6352-60). The effect on stability and oligomerization state of a model coiled coil, by substituting various amino acids at the “a” position and at the “d” position, have been reported previously, wherein formation of a trimeric structure was particularly dependent on the substitution at the “d” position (see, e.g., Tripet et al., 2000, J. Mol. Biol. 300:377-402; and Wagschal et al., 1999, J. Mol. Biol. 285:785-803). With respect to peptides derived from the native sequence of the HR1 region of HIV gp41, such peptides self assemble predominantly in tetrameric form at higher concentrations of peptide, and destabilize into monomers at lower concentrations (nanomolar range or less) of peptide.

A synthetic peptide of the present invention comprises the following distinguishing and functional characteristics. A. Sequence.

A synthetic peptide according to the present invention is derived from the native sequence of the HR1 region of HIV-1 gp41 in that it comprises a sequence that includes a series of contiguous amino acids that are identical to a series of contiguous amino acids of the HR1 region (preferably, no less than 16 and no more than 60 amino acids of the HR1 region), except for one or more strategically placed amino acid substitutions which unexpectedly affect the oligomerization of the peptide such that the synthetic peptide self assembles into predominantly trimeric form when in solution. A discovery of the present invention is that there is a cluster of hydrophobic amino acids in the HIV-1 gp41 HR1 region, where one or more amino acid substitutions therein results in a change in oligomerization in forming a synthetic peptide according to the present invention. This cluster of hydrophobic amino acids is characterized by two adjoining heptad repeats in which an amino acid substitution in the “g” position of the first heptad (closer to the N-terminal portion of the HR1 peptide as compared to the heptad it adjoins) and an amino acid substitution in the “c” position of the second heptad of the two adjoining heptads unexpectedly affects the oligomerization state of the synthetic peptide comprising such substitutions, as compared to an HR1 peptide of native sequence (e.g., without such substitutions). In another example, an amino acid substitution in such “c” position alone unexpectedly results in a change in the oligomerization state of a synthetic peptide comprising the substitution, as compared to an HR1 peptide of native sequence (e.g., without such substitution).

More particularly, and with reference to FIG. 2, in two adjoining heptads (abcdefgabcdefg), the cluster of hydrophobic amino acids comprises the sequence (single letter amino acid code) “IEAQQHLLQLTVWG” (amino acid residues in positions 24 to 37 of SEQ ID NO:1, or polymorphisms thereof, see, e.g., FIG. 2 for some illustrations of HR1 amino acid sequences that differ slightly from SEQ ID NO:1 as a result of viral polymorphisms). A motif of such cluster of hydrophobic amino acids, with the motif referred to hereinafter as the “hydrophobic domain” of the HR1 region, may be generally represented by the sequence “QHLLQLTVW” (amino acid residues in positions 28 to 36 of SEQ ID NO:1 or polymorphisms thereof) and comprising heptad repeat positions “efgabcdef”, or represented by the sequence “QHXXZLTVW” comprising heptad repeat positions “efgabcdef”, where X is typically leucine or methionine; and Z is typically glutamine, but may also be another amino acid such as lysine or glutamic acid. Thus, for example, a substitution in either the “c” position of the hydrophobic domain (e.g., at amino residue 33 of SEQ ID NO:1 or polymorphisms thereof) or in both the “g” position and the “c” position of this hydrophobic domain of the HR1 region (e.g., at amino acid residue 30 and amino residue 33, respectively, of SEQ ID NO:1 or polymorphisms thereof) alters the oligimerization state of the resultant synthetic peptide when in solution. It will be apparent to one skilled in the art that any peptide derived from the native sequence of the HR1 region of HIV gp41 which has antiviral activity (as can be determined using methods standard in the art without undue experimentation), and which contains all or a portion (e.g., no less than “QHLL” or “QHXX” at the carboxy terminus of the native sequence) of the hydrophobic domain, can be used as a native sequence into which one or more amino acid substitutions in the hydrophobic domain may be introduced to produce a synthetic peptide according to the present invention. For purposes of illustration, such HR1 peptides derived from the native sequence, and from which a synthetic peptide may be produced, may include, but are not limited to, SEQ ID NOs:1 to 28.

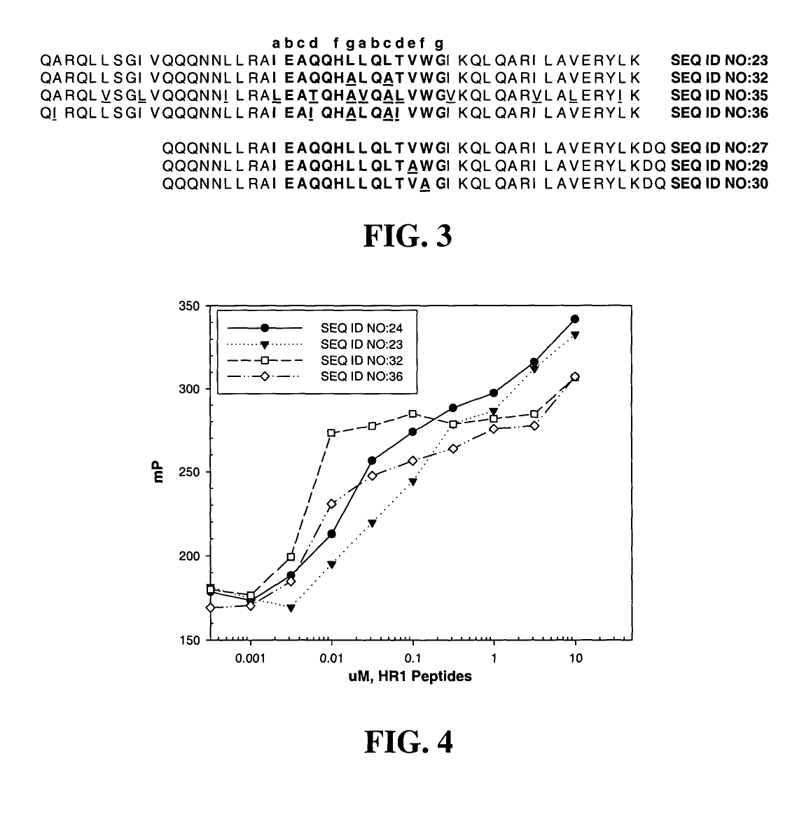

For example, a synthetic peptide according to the present invention comprises a heptad repeat that differs from the native sequence of a heptad repeat of the HR1 region of HIV-1 gp41 in that in the hydrophobic domain of the synthetic peptide according to the present invention, there is an amino acid substitution in either the “c” position, or in both the “g” position and the “c” position. More particularly, it is noted with significance that native sequences of the HR1 region of gp41 appear strikingly conserved in having a bulky, buried, nonpolar amino acid consisting of either a leucine or methionine in the “g” position of the hydrophobic domain and a leucine in the “c” position of the hydrophobic domain (see, e.g., FIG. 2). The synthetic peptides of the present invention, as compared to the native sequence, comprises a substitution in either the “c” position of the hydrophobic domain or in both the “g” position and “c” position of the hydrophobic domain, with an amino acid, preferably with a hydrophobic amino acid other than methionine and leucine, more preferably with a hydrophobic amino acid with uncharged, nonpolar head groups (e.g., alanine, valine, and the like), and more preferably with an amino acid comprising alanine. Thus, the synthetic peptide comprises a heptad repeat containing this hydrophobic domain and one or more amino acid substitutions therein (at the “c” position or at both the “g” and “c” positions), which one or more amino acid substitutions confers on the synthetic peptide the ability to self-assemble in solution to a predominately trimeric form as can be determined using methods standard in the art (see, e.g., Example 1 and Table 1). The number of heptads in the heptad repeat may vary, depending on the length of the synthetic peptide according to the present invention. However, preferably, the synthetic peptide will comprise a heptad repeat comprised of at least 3 complete, contiguous heptads, and more preferably will comprise a heptad repeat of at least 5 complete, contiguous heptads (e.g., see FIG. 3). A synthetic peptide according to the present invention, in addition to the one or more substitutions at the “g” position and/or “c” position of the hydrophobic domain, may also comprise an additional amino acid substitution in one or more of the heptads of the peptide in a position selected from the group consisting of an “a” positions, a “d” position, a “b” position, or a combination thereof (see, e.g., FIG. 3, and Example 2), as compared to the native sequence of HR1 of HIV-1 gp41. As illustrated herein, such additional amino acid substitutions may range in number from about 1 to about 15 amino acid substitutions, with the synthetic peptide still retaining the ability to self-associate in solution into trimers, and increased antiviral activity against HIV-1, as compared to native sequence.

In another embodiment, a synthetic peptide according to the invention may comprise an amino acid substitution in either or both of the C-terminal “e” position and “f” position of the hydrophobic domain of the HR1 region (e.g., amino acid residue 35 and/or amino 36 of SEQ ID NO:1 or a polymorphism thereof); comprising a substitution of either or both of the valine and tryptophan (see, e.g., FIG. 3, SEQ ID NO:29-30) as compared to the native sequence of the HR1 region of HIV-1 gp41. It is noted with significance, that amino acids at these two positions (e.g., the valine and tryptophan) appear to be highly conserved amongst different strains of HIV-1 (see, e.g., FIG. 2). As compared to the native sequence, a synthetic peptide of the present invention comprises a substitution in either or both the C terminal “e” position and “f” position of the hydrophobic domain with an amino acid, preferably with a hydrophobic amino acid other than valine (with respect to the “e” position) and tryptophan (with respect to the “f” position), more preferably with a hydrophobic amino acid with uncharged, nonpolar head groups (e.g., alanine and the like), and more preferably with alanine, which amino acid substitution(s) confers on the synthetic peptide the ability to self-assemble in solution to a predominately trimeric form as can be determined using methods standard in the art. In a preferred embodiment, and as compared to the native sequence, a synthetic peptide according to the present invention comprises a combination of amino acid substitutions as described in this section A (e.g., a substitution in the “c” position or in both the “g” and “c” position, and in one or more of the C-terminal “e” position and ‘T’ position, in the hydrophobic domain; a substitution in the “c” position or in both the “g” and “c” position, and in one or more of the C-terminal “e” position and “f” position, of the hydrophobic domain, and further comprising substitutions in one or more of the heptads of the peptide in one or more of an “a” position, “b” position, and “d” position; a substitution in either or both the C-terminal “e” position and “f” position of the hydrophobic domain, and further comprising substitutions in one or more of the heptads of the peptides in one or more of an “a” position, a “b” position, and a “d” positions; etc.).

B. Oligomerization

HR1 peptides, peptides derived from the HR1 region of HIV gp41, have been reported to exist in solution in a monomer/dimer/tetramer equilibrium, and can self-assemble into predominately tetramers, depending on the concentration of the peptide. Since the synthetic peptides according to the present invention can self-assemble into trimers (the preferred oligomeric form upon self-assembly), a primary advantage of the synthetic peptides and methods of the present invention (as compared to previously described HR1 peptides) is that a native-like trimeric core formed by a synthetic peptide according to the present invention is presented to interact with and bind to the trimeric HR2 core of HIV-1 gp41 in blocking the binding interactions between the native trimeric HR1 core with the trimeric HR2 core of HIV-1 gp41 necessary for HIV-1 gp41-mediated fusion to occur. Thus, the ability of a synthetic peptide according to the present invention to self assemble in solution to predominately trimers is an improved and unexpected function in comparison to HR1 peptides previously described in the art. Since known peptide models for HR1 regions focus on the “a” position and “d” position in the heptad repeat as being the determinants that determine oligomerization, it is unexpected that a peptide can preferably self-assemble in solution as trimers as a result of a substitution in either the “c” position or in both the “g” position and the “c” position of the hydrophobic domain, and/or in one or more of the C-terminal “e” position and “f” position of the hydrophobic domain, of the native sequence of the HR1 region of HIV-1 gp41. The discovery made by the present inventors is that there are specific, key residues in the amino acid sequence of the HR1 region of HIV-1 gp41 (other than at the “a” and “d” positions) that may govern oligomerization; and further, that substitution of one or several of these key residues can result in a synthetic peptide that self-assembles in solution into trimers as a preferred oligomeric form (e.g., as opposed to a tetramer). A trimer of the present invention is formed in solution (e.g., in a physiological solution or other aqueous environment) by self-assembly of synthetic peptide according to the present invention. C. Size A synthetic peptide according to the present invention may comprise a sequence of no less than about 18 amino acids and no more than about 60 amino acid residues in length, and preferably no less than 30 amino acids and no more than about 51 amino acids in length, and more preferably no less than about 41 amino acids and no more than about 51 amino acids in length. Preferably, a synthetic peptide according to the present invention comprises a contiguous sequence consisting of at least 20 amino acid residues within the amino acid residues 23 to 59 of SEQ ID NO:1, and more preferably comprises amino acids “IEAQQHLLQLTVWG” (e.g., amino acids 24 to 37 of SEQ ID NO:1; or polymorphisms thereof), and further comprising one or more amino acid substitutions in the hydrophobic domain thereof, as this portion of the HR1 region and such substitutions in the hydrophobic domain have been found pursuant to this invention to influence the oligomeric form described herein in more detail. As also described herein in more detail, a synthetic peptide according to the present invention may further comprise a macromolecular carrier.

D. Ability to Bind to HR2 Peptides

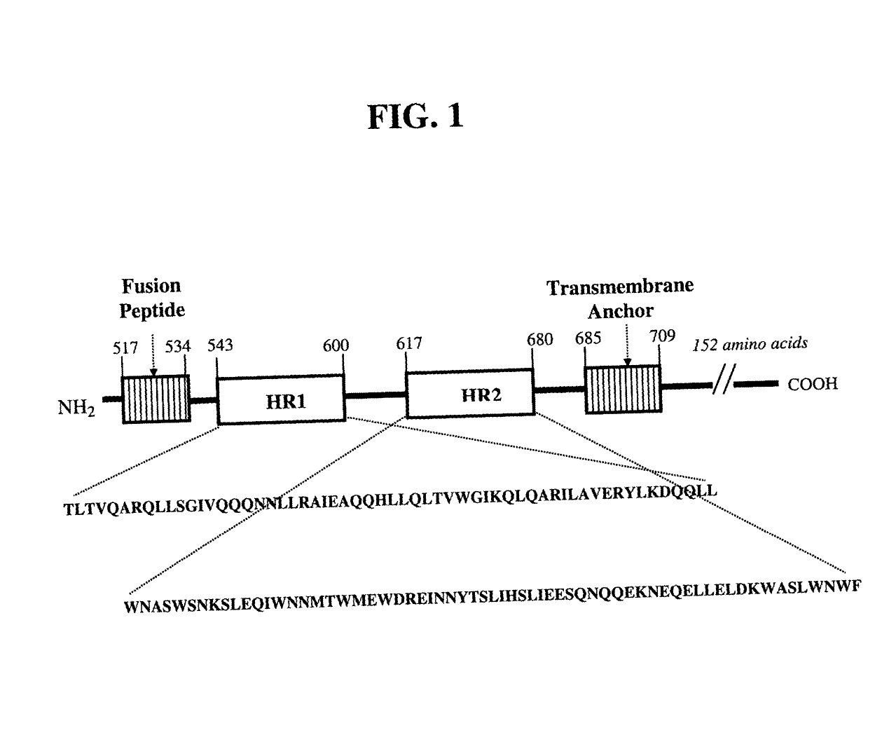

It is an important feature of each of the synthetic peptides according to the present invention (preferably in self-assembled trimeric form) to be capable of complexing with the HIV-1 gp41 HR2 region (and as demonstrated by a peptide derived from the HR2 region). Peptides derived from the HR2 region (“HR2 peptides”) that are known to complex with HR1 amino acid sequences are well known in the art (see, e.g., U.S. Pat. No. 5,464,933; see also FIG. 1). More specifically, in solution a synthetic peptide according to the present invention self-assembles into trimers, and such trimers are capable of complexing with such HR2 peptide and also with the HR2 region of HIV-1 gp41. Such complexing can be detected using methods standard in the art, and as described herein in more detail.

As described herein in more detail, a synthetic peptide according to the present invention may further comprise a component comprising: (a) one or more reactive functionalities (e.g., at either the C-terminal end, or N-terminal end, or a combination thereof (both the C-terminal end and N-terminal end)); or (b) a pharmaceutically acceptable carrier; or (c) a macromolecular carrier; or (d) an amino acid substitution comprising an addition of no less than one amino acid and no more than twenty amino acids to either or both of the amino terminus or carboxy terminus of the synthetic peptide; or (e) a combination thereof. Likewise, a trimer according to the present invention may further comprise a component comprising: (a) one or more reactive functionalities; or (b) a pharmaceutically acceptable carrier; or (c) a macromolecular carrier; or (d) an amino acid substitution comprising an addition of no less than one amino acid and no more than twenty amino acids to either or both of the amino terminus or carboxy terminus of synthetic peptide forming the trimer; or (e) a combination thereof. The present invention is illustrated in the following examples, which are not intended to be limiting.

### EXAMPLE 1

Peptides were synthesized on a peptide synthesizer using standard solid-phase synthesis techniques and using standard FMOC peptide chemistry. In this example, the synthetic peptides further comprised reactive functionalities; i.e., were blocked at the N-terminus by an acetyl group and at the C-terminus by an amide group. After cleavage from the resin, the peptides were precipitated, and the precipitate was lyophilized. The peptides were then purified using reverse-phase high performance liquid chromatography; and peptide identity was confirmed with electrospray mass spectrometry.

In one embodiment, synthetic peptide, as exemplified by the synthetic peptide having the amino acid sequence of SEQ ID NO:32, was synthesized to comprise amino acid substitutions at the “g” position and “c” position in the hydrophobic domain. The synthetic peptide having the amino acid sequence of SEQ ID NO:32 was derived from the HR1 region of HIV-1 gp41 in that it comprises a sequence corresponding to amino acid residues 5 to 53 of SEQ ID NO:1 except, however, amino acid substitutions were made in amino acid residues corresponding to the “g” position and the “c” position of the hydrophobic domain (each a leucine in the HIVIIIB sequence). Thus, the synthetic peptide having the amino acid sequence of SEQ ID NO:32 is 49 amino acids in length, and comprises a heptad repeat of 6 complete heptads with 5 leucine zipper-like motifs. A similar embodiment of the present invention is a synthetic peptide having the amino acid sequence of SEQ ID NO:34, which has the same sequence as the synthetic peptide having the amino acid sequence of SEQ ID NO:32 except, however, that two amino acids (an aspartic acid and glutamine) were added to the C-terminus. In yet another illustration of the present invention, synthetic peptide, as exemplified by the synthetic peptide having the amino acid sequence of SEQ ID NO:81, was synthesized to comprise amino acid substitution at only the “c” position in the hydrophobic domain. The synthetic peptide having the amino acid sequence of SEQ ID NO:81 was derived from the HR1 region of HIV-1 gp41 in that it comprises a sequence corresponding to amino acid residues 5 to 55 of SEQ ID NO:1 except, however, amino acid substitution was made in amino acid residue corresponding to the “c” position of the hydrophobic domain (amino acid residue in position 33 of SEQ ID NO:1; e.g., a leucine in the HIVIIIB sequence). Thus, the synthetic peptide having the amino acid sequence of SEQ ID NO:81 is 51 amino acids in length, and comprises a heptad repeat of 6 complete heptads with 5 leucine zipper-like motifs. A similar embodiment of the present invention is a synthetic peptide having the amino acid sequence of SEQ ID NO:82, and comprising a sequence corresponding to amino acid residues 15 to 55 of SEQ ID NO:1 except, however, amino acid substitution was made in amino acid residue corresponding to the “c” position of the hydrophobic domain (amino acid residue in position 33 of SEQ ID NO:1; e.g., a leucine in the HIV_{LAI} sequence). These synthetic peptides were compared to an HR1 peptide, derived from native sequence of the HR1 region and which has the amino acid sequence of SEQ ID NO:23, for oligomerization state, helicity, stability, and binding to HR2 peptide (such as an HR2 peptide having the amino acid sequence of SEQ ID NO:33) using the following methods.

Oligomerization state was assessed by using methods standard in the art for performing sedimentation equilibrium experiments and for analysis of the resultant data. In that regard, sedimentation equilibrium experiments were performed over a concentration range of synthetic peptide (1 μM to 1001M) as follows. Briefly, six-channel cells (12 mm optical path length) were used with an An-60 Ti rotor operated at 13,500 rpm, 18,000 rpm, 22,000 rpm, and 35,000 rpm) in an analytical ultracentrifuge at 4° C. The cell radii were scanned using 0.001 cm steps with 10 averages/scan at 3 different wavelengths (235 nm, 240 nm, 280 nm). Data obtained with solutions containing synthetic peptide was analyzed using a single species model to determine a weight-averaged molecular weight (MW). Diagnostic plots of M_{W}/M_{W0} vs. rpm/rpm₀ and M_{W} vs. radial concentration were used to test for sample homogeneity. When systemic residuals or a M_{W} higher than monomer molecular weight indicated the presence of self-association, associative models were investigated. The suitability of a model (goodness of fit) was judged by the trends observed in the residuals.

Helicity was assessed by circular dichroism (“CD”) as follows. Briefly, CD spectra were obtained using a spectrometer equipped with a thermoelectric temperature controller. The spectra was obtained at 25° C. with 0.5 nanometer (nm) steps from 200 to 260 nm, with a 1.5 nm bandwith, and a typical averaging time of 4 seconds/step. After the cell/buffer blank was subtracted, spectra were smoothed using a third-order least-squares polynomial fit with a conservative window size to give random residuals. Raw ellipticity values were converted to mean residue ellipticity using standard methods, and plotted was the wavelength (from 200 to 260 nm) versus [θ]×10⁻³ (degrees cm²/dmol). Percent helicity values were then calculated using standard methods. Assessment of thermal stability was performed by monitoring the change in CD signal at 222 nm as temperature was raised in 2° C. steps, with 1 minute equilibration times. The thermal stability for each sample (e.g., synthetic peptide) was represented by the transition temperature (Tm) determined by the derivative method.

In determining antiviral activity (e.g., one measure being the ability to inhibit transmission of HIV to a target cell) of the synthetic peptides according to the present invention, used was an in vitro assay which has been shown, by data generated using peptides derived from the HR regions of HIV gp41, to be predictive of antiviral activity observed in vivo. More particularly, antiviral activity observed using an in vitro infectivity assay (“Magi-CCR5 infectivity assay”; see, e.g., U.S. Pat. No. 6,258,782) has been shown to reasonably correlate to antiviral activity observed in vivo for the same HIV gp41 derived peptides (see, e.g., Kilby et al., 1998, Nature Med. 4:1302-1307). These assays score for reduction of infectious virus titer employing the indicator cell lines MAGI or the CCR5 expressing derivative cMAGI. Both cell lines exploit the ability of HIV-1 tat to transactivate the expression of a β-galactosidase reporter gene driven by the HIV-LTR. The β-gal reporter has been modified to localize in the nucleus and can be detected with the X-gal substrate as intense nuclear staining within a few days of infection. The number of stained nuclei can thus be interpreted as equal to the number of infectious virions in the challenge inoculum if there is only one round of infection prior to staining. Infected cells are enumerated using a CCD-imager and both primary and laboratory adapted isolates show a linear relationship between virus input and the number of infected cells visualized by the imager. In the MAGI and cMAGI assays, a 50% reduction in infectious titer (Vn/Vo=0.5) is significant, and provides the primary cutoff value for assessing antiviral activity (“IC50” is defined as the dilution resulting in a 50% reduction in infectious virus titer). A secondary cutoff of Vn/Vo=0.1, corresponding to a 90% reduction in infectious titer is also assessed (“IC90”). Synthetic peptides tested for antiviral activity were diluted into various concentrations, and tested in duplicate or triplicate against an HIV inoculum adjusted to yield approximately 1500-2000 infected cells/well of a 48 well microtiter plate. The synthetic peptide (in the respective dilution) was added to the cMAGI or MAGI cells, followed by the virus inocula; and 24 hours later, an inhibitor of infection and cell-cell fusion (e.g., T-20) was added to prevent secondary rounds of HIV infection and cell-cell virus spread. The cells were cultured for 2 more days, and then fixed and stained with the X-gal substrate to detect HIV-infected cells. The number of infected cells for each control and peptide dilution was determined with the CCD-imager, and then the IC50 and IC90 were calculated (expressed in (μg/ml).

Several assays were used to assess the ability of the synthetic peptides/trimers according to the present invention to retain the ability to complex with peptides derived from the HR2 region of HIV-1 gp41. These include a protein chip assay (using Biacore analysis) in which surface plasmon resonance is used to detect the binding, and kinetics thereof, between a synthetic peptide and an HR2 peptide; and a fluorescence polarization assay. In the fluorescence polarization assay, a detectable moiety comprising a fluorescent dye (e.g., rhodamine and/or fluorescein) was used to label HR2 peptide, and the assay was then allowed to proceed under sufficient conditions and for a sufficient period of time for labeled peptide to bind to HR1 peptide, or synthetic peptide (or trimer formed therefrom). Fluorescence intensity of polarized light was then measured with a detection system comprising a spectrophotometer, and the amount of polarized light was calculated (units=mP). As illustrated in FIG. 4, and exemplified by synthetic peptide having the amino acid sequence of SEQ ID NO:32, the synthetic peptides according to the present invention (or trimers formed therefrom) retain the ability to complex with peptides derived from the HR2 region of HIV-1 gp41. In some instances throughout the Examples illustrated herein, such ability to complex with HR2 peptides as demonstrated by this binding assay is denoted as “yes” with reference to such binding activity.

Comparison of synthetic peptides (illustrated as SEQ ID NO:32 and SEQ ID NO:34) with HR1 peptides of respective native sequence (illustrated as SEQ ID NO:23 and SEQ ID NO:24) is illustrated in Table 1.

TABLE 1
Biophysical and antiviral Parameters

| | SEQ ID | SEQ ID | SEQ ID | SEQ ID | SEQ ID | SEQ ID |
|---|---|---|---|---|---|---|
| Parameter | NO: 23 | NO: 24 | NO: 32 | NO: 34 | NO: 81 | NO: 82 |
| Oligomeri- | Aggregates | Best | Best | Best | Best | Best |
| zation | | modeled | modeled | modeled | modeled | modeled |
| | | As a | as a | as a | as a | as a |
| | | tetramer | trimeric | trimeric | trimeric | trimeric |
| | | | species* | species* | species* | species* |
| Helicity | 83% | 74% | 87% | 100% | 68% | 72% |
| (at 10 μM, | | | | | | |
| 25° C.) | | | | | | |
| Stability | Tₘ 83° C. | Tₘ 82° C. | Tₘ 69° C. | Tₘ 71° C. | Tₘ 77° C. | Tₘ 68° C. |
| (at 10 μM) | | | | | | |
| HR2 peptide | Yes | yes; 1.2 × 10⁶/ | Yes; 6.1 × 10⁵/ | Yes | Yes | Yes |
| binding | | 1.2 × 10⁻³/ | 1.9 × 10⁻³/ | | | |
| K on/K off/Kd | | 1.1 nM | 3.1 nM | | | |
| Antiviral | | | | | | |
| activity | | | | | | |
| Against | | | | | | |
| HIV IIIB | | | | | | |
| (μg/ml) IC50 | 4.69 | 3.73 | 0.59 | 0.19 | 0.61 | 0.89 |
| IC90 | 23.24 | 12.301 | 1.90 | 0.62 | | |

*with only a small fraction (e.g., about 1 to about 10%) of higher order oligomer (e.g., hexamer) and therefore is considered to self assemble into trimers.

As shown in Table 1, as compared to a respective peptide having the native sequence of HR1 (e.g., SEQ ID NOs: 23 & 24), a synthetic peptide according to the present invention (e.g., as exemplified by SEQ ID NOs:32, 34, 81, and 82) resulted in an alteration in the oligomerization state. Additionally, as shown in Table 1 (and subsequent Tables herein), a synthetic peptide according to the present invention (e.g., trimers formed therefrom) can demonstrate relatively good binding to HR2 peptide, and a significant increase in antiviral activity (e.g., 3 fold or greater increase in potency, as observed by a ⅓ or greater decrease in either or both of IC50 and IC90) as compared to a respective HR1 peptide of native sequence. It was an unexpected result that by replacing the leucine at the aforementioned “c” position of the hydrophobic domain (e.g., as exemplified by synthetic peptides having the amino acid sequence of SEQ ID NOs:81 & 82), or replacing the leucine or methionine at the aforementioned “g” position of the hydrophobic domain and the leucine at the aforementioned “c” position of the hydrophobic domain (e.g., as exemplified by synthetic peptides having the amino acid sequence of SEQ ID NOs:32 & 34) with a less bulky amino acid (an amino acid having a less bulky side chain or R group than leucine or methionine), altered in the synthetic peptide derived from the HR1 region can be (a) the oligomerization state of such synthetic peptide, in that it can self-assemble in solution to predominately trimers rather than tetramers (e.g., the latter being the predominant oligomeric state in solution of respective native sequence having the amino acid sequences of SEQ ID NOs:23 & 24); and (b) antiviral activity to a significant improvement (increase) in antiviral activity against HIV isolates (e.g., compare the statistically significant difference in the antiviral activity of a trimer of a synthetic peptide having the amino acid sequence of any one of SEQ ID NOs:32, 34, 81 or 82 versus a tetramer of an HR1 peptide of native sequence shown in SEQ ID NO:24). Amino acid residues which are less bulky than leucine and methionine are known to those skilled in the art to include, but are not limited to, alanine, glycine, valine, serine, threonine, and the like.

### EXAMPLE 2

In another embodiment, synthetic peptides, exemplified by the peptides having the amino acid sequences of SEQ ID NOs:35-40, were synthesized using the methods outlined in Example 1 herein to comprise amino acid substitutions at the “g” position and “c” position in the hydrophobic domain of the HR1 region of HIV-1 gp41, and additionally comprised an amino acid substitution in either (a) the “a” position of at least one of the heptads of the plurality of heptads comprising the peptide; or (b) an amino acid substitution in the “d” position of at least one of the heptads of the plurality of heptads comprising the peptide; or (c) a combination thereof (i.e., a combination of (a) and (b)). Where there is the combination thereof, the substitution in the “a” position and the substitution in the “d” position may be in the same heptad of the peptide, and/or may be in a different heptad of the peptide. In a preferred embodiment, the substitution in either the “a” position and/or the “d” position is with an amino acid that are known to the art to stabilize the structure of a synthetic peptide; i.e., that induce the formation of a specific secondary structure (a coiled coil oligomer, for example; e.g., substitution of an amino acid other than Leu or Ile with Leu or Ile in forming additional leucine zipper-like motifs as compared to native sequence before such amino acid substitution).

A. This example illustrates a synthetic peptide according to the present invention which, in addition to having amino acid substitutions in the “g” and “c” positions of the hydrophobic domain of the HR1 region of HIV-1 gp41, comprises an amino acid substitution in the “a” position of at least one of the heptads of the plurality of heptads comprising the peptide, and comprises an amino acid substitution in the “d” position of at least one of the heptads of the plurality of heptads comprising the peptide. For example, a synthetic peptide having the amino acid sequence of SEQ ID NO:35 comprises the same amino acid sequence as a synthetic peptide having the amino acid sequence of SEQ ID NO:32, except, however, amino acid substitutions were made in amino acids corresponding to residue positions 6, 20, 27, 34, 41, and 48 (e.g., in the “a” position of at least one heptad of the plurality of heptads), and to residue positions 9, 16, 23, 30, and 44 (e.g., in the “d” position of at least one heptad of the plurality of heptads), of SEQ ID NO:32. Thus, the synthetic peptide having the amino acid sequence of SEQ ID NO:35 comprises 49 amino acids in length and comprises a heptad repeat of 6 complete heptads but with only 3 leucine zipper-like motifs (as compared to 5 leucine zipper-like motifs for SEQ ID NO:32). Unexpectedly, the peptide having the amino acid sequence of SEQ ID NO:35 demonstrates a higher degree of stability as compared to that of the synthetic peptide having the amino acid sequence of SEQ ID NO:32 (see, Table 2).

In another example, the synthetic peptide having the amino acid sequence of SEQ ID NO:37 comprises the same amino acid sequence as the synthetic peptide with the amino acid sequence of SEQ ID NO:35 (an amino acid substitution in one or more “a” positions and in one or more “d” positions) except for one additional substitution at amino acid residue position 35 of SEQ ID NO:35 (e.g., in a “b” position of a heptad, whereby the arginine is substituted for a lysine). Thus, the synthetic peptide having the amino acid sequence of SEQ ID NO:37 comprises 49 amino acids in length and comprises a heptad repeat of 6 complete heptads and 3 leucine zipper-like motifs. Although synthetic peptides having the respective amino acid sequence of SEQ ID NO:35 and 37 vary by only one residue, as shown in Table 2, the helicity and stability are increased when the arginine residue is substituted for the lysine residue.

In another example, the synthetic peptide having the amino acid sequence of SEQ ID NO:38 comprises the same amino acid sequence as SEQ ID NO:32, except, however, substitutions were made in amino acid residue positions corresponding to residues 27, 34, 41, and 48 (e.g., in the “a” position of at least one heptad in the plurality of heptads) as well as amino acid residue positions corresponding to residues 23, 30, and 44 (e.g. in the “d” position of at least one heptad in the plurality of heptads), of SEQ ID NO:32. In addition, two amino acid residues, an aspartic acid and a glutamine, were added to the C-terminus, so that the synthetic peptide having the amino acid sequence of SEQ ID NO:38 comprises 51 amino acid residues. This synthetic peptide comprises a heptad repeat of 6 complete heptads and 3 leucine zipper-like motifs.

In yet another example, the synthetic peptide having the amino acid sequence of SEQ ID NO:39 has the same amino acid sequence as the synthetic peptide having the amino acid sequence of SEQ ID NO:32, except, however, substitutions were made at the amino acid residue positions corresponding to residues 6, 20, 41, and 48 (e.g., in the “a” position of at least one heptad in a plurality of heptads), and in amino acid residue positions corresponding to residues 9, 16, and 44 (e.g., in the “d” position of at least one heptad in a plurality of heptads) of SEQ ID NO:32; and additionally, an aspartic acid and glutamine were added to the C-terminus. Thus, the synthetic peptide having the amino acid sequence of SEQ ID NO:39 comprises a total length of 51 amino acid residues with 6 complete heptads and 4 leucine zipper-like motifs.

Such illustrative synthetic peptides (having the respective amino acid sequences of SEQ ID NOs:35, and 37-39) were compared to a peptide derived from that native sequence of the HR1 region without any amino acid substitutions (see, FIG. 3, SEQ ID NO:23) for oligomerization state, helicity, stability, antiviral activity, and binding to HR2 peptide (e.g., SEQ ID NO:33) using methods as previously described in more detail in Example 1 herein. The comparisons of these illustrative synthetic peptides according to the present invention to an HR1 peptide of native sequence are illustrated in Table 2.

TABLE 2
Biophysical and antiviral Parameters

| | SEQ ID | SEQ ID | SEQ ID | SEQ ID | SEQ ID |
|---|---|---|---|---|---|
| Parameter | NO: 23 | NO: 35 | NO: 37 | NO: 38 | NO: 39 |
| Oligomeri- | Aggregates | Trimer | Trimer* | Trimer* | Trimer* |
| zation | | | | | |
| Helicity | 83% | 78% | 85% | 79% | 63% |
| (10 μM, | | | | | |
| 25° C.) | | | | | |
| Stability | Tₘ 83° C. | Tₘ >97° C. | Tₘ > | Tₘ | Tₘ |
| (at 10 μM) | | | 97° C. | 90° C. | 68° C. |
| HR2 peptide | Yes | Yes | Yes | Yes | Yes |
| Binding | | 1.5 × 10⁶/ | | | |
| K on/K off/Kd | | 0.1 × 10⁻³/ | | | |
| | | 21.8 nM | | | |
| Antiviral | | | | | |
| activity | | | | | |
| against | | | | | |
| HIV IIIB | | | | | |
| IC50 | 4.69 | 0.14 | <0.78 | 0.18 | 0.26 |
| IC90 (μg/ml) | 23.24 | 0.69 | | 0.88 | 1.20 |

*Best modeled as a trimer with only a small fraction (e.g., about 1 to about 10%) of higher order oligomer (e.g., hexamer), and therefore, is considered to self assemble predominately into trimers.

B. This example illustrates a synthetic peptide according to the present invention which, in addition to having amino acid substitutions in the “g” and “c” positions of the hydrophobic domain of the HR1 region of HIV-1 gp41, comprises an amino acid substitution in the “d” position of at least one of the heptads of the plurality of heptads comprising the peptide. Illustrating this example, a synthetic peptide having the amino acid sequence of SEQ ID NO:36 comprises the same amino acid sequence as SEQ ID NO:32, except, however, amino acid substitutions were made in amino acid residue positions corresponding to residues 2, 23, and 30 (e.g., in the “d” position of at least one heptad of the plurality of heptads) of SEQ ID NO:32. Thus, the synthetic peptide having the amino acid sequence of SEQ ID NO:36 comprises 49 amino acids in length and comprises a heptad repeat of 6 complete heptads but with at least 6 leucine zipper-like motifs (as compared to 5 leucine zipper-like motifs for the synthetic peptide having the amino acid sequence of SEQ ID NO:32 and 3 leucine zipper-like motifs for the synthetic peptide having the amino acid sequence of SEQ ID NO:35). Such illustrative synthetic peptide (having the amino acid sequence of SEQ ID NO:36) was compared to a peptide derived from that native sequence of the HR1 region without any amino acid substitutions (SEQ ID NO:23) for oligomerization state, helicity, stability, antiviral activity, and binding to HR2 peptide, using methods as previously described in more detail in Example 1 herein. The comparisons of this illustrative synthetic peptide according to the present invention to a peptide of native sequence is illustrated in Table 3.

TABLE 3
Biophysical and antiviral Parameters

| Parameter | SEQ ID NO: 23 | SEQ ID NO: 36 |
|---|---|---|
| Oligomerization | Aggregates | Trimer* |
| Helicity (10 μM, 25° C.) | 83% | 90% |
| Stability (at 10 μM) | Tₘ 83° C. | Tₘ > 97° C. |
| HR2 peptide binding | Yes | Yes |
| K on/K off/Kd | | 4.3 × 10⁵/3.3 × 10⁻³/ |
| | | 7.7 nM |
| Antiviral activity | | |
| against HIV IIIB | | |
| IC50 | 4.69 | 0.73 |
| (μg/ml) IC90 | 23.24 | 3.03 |

*as determined by crystallography

C. This example illustrates a synthetic peptide according to the present invention which, in addition to having amino acid substitutions in the “g” and “c” positions of the hydrophobic domain of the HR1 region of HIV-1 gp41, comprises an amino acid substitution in the “a” position of at least one of the heptads of the plurality of heptads comprising the peptide. Illustrating this example, a synthetic peptide having the amino acid sequence of SEQ ID NO:40 comprises the same amino acid sequence of the peptide having the amino acid sequence of SEQ ID NO:32, except, however, that a substitution was made in amino acid residue position corresponding to residue 48 (e.g., in the “a” position of at least one heptad in a plurality of heptads) of SEQ ID NO:32; and additionally, an aspartic acid and glutamine were added to the C-terminus of the peptide. Thus, the synthetic peptide having an amino acid sequence of SEQ ID NO:40 comprises a total length of 51 amino acid residues, with 6 complete heptads and 3 leucine zipper-like motifs. Such illustrative synthetic peptide (having the amino acid sequence of SEQ ID NO:40) was compared to a peptide derived from that native sequence of the HR1 region without any amino acid substitutions (SEQ ID NO:23) for oligomerization state, helicity, stability, antiviral activity, and binding to HR2 peptide, using methods as previously described in more detail in Example 1 herein. The comparisons of this illustrative synthetic peptide according to the present invention to a peptide of native sequence is illustrated in Table 4.

TABLE 4
Biophysical and Antiviral Parameters

| Parameter | SEQ ID NO: 23 | SEQ ID NO: 40 |
|---|---|---|
| Oligomerization | Aggregates | Trimer* |
| Helicity (at 10 μM) | 83% | 53% |
| Stability(at 10 μM) | Tₘ 83° C. | Tₘ 62° C. |
| HR2 peptide binding | Yes | Yes |
| Antiviral activity | | |
| against HIV IIIB | | |
| IC50 | 4.69 | 0.41 |
| (μg/ml) IC90 | 23.24 | 1.84 |

*with only a small fraction (e.g., about 1 to about 10%) of higher order oligomer (e.g., hexamer) and therefore is considered to self assemble into trimers.

As shown in Tables 2, 3, and 4, as compared to a peptide of the native sequence of HR1 (as illustrated by SEQ ID NO:23), a synthetic peptide according to the present invention resulted in an alteration in the oligomerization state to predominately a trimer in solution, while retaining binding to HR2 peptide, and can demonstrate a significant improvement in antiviral activity (e.g., at least a 5 fold increase in potency). As illustrated by synthetic peptides having amino acid sequences of SEQ ID NOs: 35 and 36, the data clearly indicates that the synthetic peptides can self-assemble into trimers (conclusion based on current structural data) and that the trimers are in a coiled coil (conclusion based on stability data). Such coiled coils model the trimeric coiled coils of the HR1 region of HIV gp41, and such coiled coils produced by the synthetic peptides according to the present invention also retain HIV gp41 HR2 binding activity (see, e.g., FIG. 4, SEQ ID NO:36). Also note in comparing the results shown in Tables 2 and 3, a synthetic peptide which additionally had a substitution in the “d” position of at least one of the heptads comprising the synthetic peptide unexpectedly resulted in a significantly improved helicity and stability.

In the example of the synthetic peptide having the amino acid sequence of SEQ ID NO:35, generally speaking, the majority of amino acid substitutions in the “a” position and “d” position were either leucine for isoleucine, or isoleucine for leucine, or a less bulky amino acid (e.g., valine) for either isoleucine or leucine. In comparing Table 1 with Table 2, and the biochemical and antiviral properties of the synthetic peptide having the amino acid sequence of SEQ ID NO:32 with the synthetic peptide having the amino acid sequence of SEQ ID NO:35, it was an unexpected result that by making these changes in the “a” position of at least one heptad of the plurality of heptads, and in the “d” position of at least one heptad of the plurality of heptads that improved is stability of the peptide as measured by a higher Tₘ (e.g., despite the reduction in number of leucine zipper-like motifs). The three-dimensional structure of SEQ ID NO:35 was determined by X-ray crystallography using methods standard in the art. These structures revealed that (a) the synthetic peptide having the amino acid sequence of SEQ ID NO:35 self-assembles into trimers, and (b) that the synthetic peptides in the trimer adopt a secondary structure comprising a coiled coil. The findings by X-ray crystallography confirm the data generated by oligomerization studies and stability studies which indicated that a synthetic peptide according to the present invention can form trimers which are coiled coil.

D. In this example, highlighted is the importance in a synthetic peptide according to the present invention to have amino acid substitutions in the “g” and “c” positions of the hydrophobic domain of the HR1 region of HIV-1 gp41. For comparative purposes, the peptide having the amino acid sequence of SEQ ID NO:41 has the same “a” and “d” substitutions as the synthetic peptide having the amino acid sequence of SEQ ID NO:35, but lacks the substitutions in the “g” position and the substitution in the “c” position of the hydrophobic domain. Similarly, the peptide having the amino acid sequence of SEQ ID NO:42 has the same “d” substitutions as the synthetic peptide having the amino acid sequence of SEQ ID NO:36, but lacks the substitution in the “g” position and the substitution in the “c” position of the hydrophobic domain. In both cases, as illustrated in Table 5, without the substitutions in the “g” and “c” positions of the hydrophobic domain, the resultant peptides demonstrate aggregate species in solution (e.g., oligomeric state other than predominately trimers), decrease in helicity, and decrease in antiviral activity, as compared to SEQ ID NO:35 and SEQ ID NO:36 (see Tables 2 and 3, respectively). The peptide having the amino acid sequence of SEQ ID NO:42 even has less antiviral activity than SEQ ID NO:23, the peptide derived from the native sequence of HIV-1 gp41 HR1. Thus, it can be concluded that substitutions in the “g” position and “c” position of the hydrophobic domain play a key role in the unexpected results with respect to biophysical and antiviral parameters of synthetic peptides according to the present invention, and trimers formed therefrom.

TABLE 5
Biophysical and Antiviral Parameters

| | SEQ ID | SEQ ID | SEQ ID |
|---|---|---|---|
| Parameter | NO: 23 | NO: 41 | NO: 42 |
| Oligomerization | Aggregates | Soluble | Soluble |
| | | Aggregates | Aggregates |
| Helicity (at 10 μM) | 83% | 73% | 85% |
| Stability (at 10 μM) | Tₘ 83° C. | Tₘ > 97° C. | Tₘ > 97° C. |
| HR2 peptide binding | Yes | Yes | Not |
| | | | determined |
| | | | (precipitates) |
| Antiviral activity | | | |
| against HIV IIIB | | | |
| IC50 | 4.69 | 3.892 | 13.605 |
| (μg/ml) IC90 | 23.24 | 14.53 | 33.56 |

### EXAMPLE 3

In another embodiment, a synthetic peptide of the present invention comprises an amino acid substitution in either or both the C-terminal “e” position and “f” position in the hydrophobic domain of the HR1 region of the native sequence of HIV-1 gp41. The amino acid substitution in the C-terminal “e” position corresponds to amino acid residue position residue 35, and the amino acid substitution in the C-terminal “f” position corresponds to amino acid residue position residue 36, of SEQ ID NO:1 or polymorphisms thereof. Preferably, the amino acid substitution is with a hydrophobic amino acid other than valine (with respect to the “e” position) and tryptophan (with respect to the ‘f’ position), more preferably with a hydrophobic amino acid with uncharged, nonpolar head groups (e.g., alanine and the like), and more preferably with alanine. More particularly, the HR1 region comprises a cluster of hydrophobic amino acids comprising the “hydrophobic domain”, wherein an amino acid substitution for one or more of L (leucine), V (valine), and W (tryptophan) in the hydrophobic domain disrupts the cluster sufficiently for synthetic peptide to be able to preferably self-assemble into trimers rather than some predominate oligomeric form other than trimers.

Synthetic peptides according to this embodiment of the present invention are exemplified by SEQ ID NOs:29, 30, and 43 which comprise residues 15 to 55 of SEQ ID NO:1 (and hence, comprise 41 amino acids), and additionally have the amino acid substitutions as described herein. These peptides were synthesized using the methods outlined in Example 1. A synthetic peptide having the amino acid sequence of SEQ ID NO:29 is comprised of 5 contiguous, complete heptads, and comprises the same sequence as a peptide having the amino acid sequence of SEQ ID NO:27 (native sequence from HIV-1 gp41 HR1), except that there is an amino substitution in the C-terminal “e” position of the hydrophobic domain (e.g., substitution of valine with alanine). This synthetic peptide (SEQ ID NO:29) illustrates a synthetic peptide comprising a substitution in the C-terminal “e” position in the hydrophobic domain. A synthetic peptide having the amino acid sequence of SEQ ID NO:30 is comprised of 5 contiguous, complete heptads, and comprises the same sequence as the peptide having the amino acid sequence of SEQ ID NO:27, except that there is an amino substitution in the C-terminal “f” position of the hydrophobic domain, whereby the tryptophan is substituted with an alanine. This synthetic peptide (SEQ ID NO:30) illustrates a synthetic peptide comprising a substitution in the C-terminal “f” position in the hydrophobic domain. A synthetic peptide having the amino acid sequence of SEQ ID NO:43 also comprises five contiguous, complete heptads, and comprises the same sequence as peptide having the amino acid sequence of SEQ ID NO:27, except that there is an amino acid substitution in the C-terminal “f” position of the hydrophobic domain, where the tryptophan is substituted with a phenylalanine. This synthetic peptide (SEQ ID NO:43) is another illustration of a synthetic peptide comprising a substitution in the C-terminal “f” position in the hydrophobic domain.

A further example of the present invention is a synthetic peptide corresponding to amino acid residues 5 to 53 of SEQ ID NO:1 (having an amino acid sequence of SEQ ID NO:31), which possesses the same amino acid sequence as the HR1 peptide having the amino acid sequence of SEQ ID NO:23 (native sequence from HIV-1 gp41 HR1) except that a phenylalanine is substituted for the tryptophan in the C-terminal “f” position of the hydrophobic domain, and an arginine is substituted for a lysine at an amino acid residue corresponding to the “b” position of the heptad adjoining (the C-terminus of) the hydrophobic domain (e.g., corresponding to amino acid residue position 39 of SEQ ID NO:1). The synthetic peptide having the amino acid sequence of SEQ ID NO:31 comprises six complete, contiguous heptads.

Synthetic peptides having the amino acid sequences of SEQ ID NOs: 29, 30, 31 and 43, exemplary of an embodiment of synthetic peptides according to the present invention, were compared to HR1 peptide (SEQ ID NO:27) for oligomerization state, helicity, stability, and binding to HR2 peptide (SEQ ID NO:33) using methods as previously described in more detail in Example 1 herein. The comparison of synthetic peptides having the amino acid sequences of SEQ ID NOs:29, 30, 31 and 43 to HR1 peptide (SEQ ID NO:27) is illustrated in Table 6.

TABLE 6
Biophysical and antiviral Parameters

| | SEQ ID | SEQ ID | SEQ ID | SEQ ID | SEQ ID |
|---|---|---|---|---|---|
| Parameter | NO: 27 | NO: 29 | NO: 30 | NO: 31 | NO: 43 |
| Oligomeri- | Tetra- | Trimer* | Trimer* | Trimer** | Trimer** |
| zation | mer | | | | |
| Helicity | 72% | 78% | 84% | 86% | 94% |
| (10 μM, | | | | | |
| 25° C.) | | | | | |
| Stability | Tₘ | Tₘ | Tₘ 77° C. | Tₘ 70° C. | Tₘ 74° C. |
| (at 10 μM) | 76° C. | 65° C. | | | |
| HR2 peptide | Yes | Yes | Not | Yes | Yes |
| Binding | | | determined | | |
| K on/K | | | (precipitates) | | |
| off/Kd | | | | | |
| Antiviral | | | | | |
| activity | | | | | |
| Vs. HIV IIIB: | | | | | |
| IC50 | 4.69 | 2.81 | 0.37 | 0.93 | 1.34 |
| (μg/ml) IC90 | 23.24 | 5.86 | 1.00 | 3.06 | 3.81 |

*consistent with weight-averaged MW analysis
**with only a small fraction (e.g., about 1% to about 10%) of higher order oligomer (e.g., hexamer) and therefore, is considered to self assemble predominately into trimers.

It is an unexpected result that an amino acid substitution in the either of the C-terminal “e” position and/or in the C-terminal “f” position of the hydrophobic domain, confers the oligomeric state of synthetic peptide to that comprising predominately a trimer in solution (as can be concluded from the data presented in Table 6). More particularly, amino acid substitutions in amino acid residues neighboring (i.e., at the “g” position in the same or adjacent heptad) the C-terminal “e” and “f” positions of the hydrophobic domain failed to switch the oligomeric state to self-assembly into trimers (see, e.g., peptides having the amino acid sequences of SEQ ID NOs: 44-46; and each of which self-assembles into tetramers in solution). Also as shown in Table 6, an amino acid substitution in the either of the C-terminal “e” position and/or in the C-terminal “f” position of the hydrophobic domain can result in a significant increase in helicity for the synthetic peptide, as well as an increase in antiviral activity (e.g., increase in potency; at least a 3 fold increase in potency) as compared to an HR1 peptide of the native sequence (without substutions).

### EXAMPLE 4

The present invention provides for synthetic peptides (preferably when in trimeric form), derived from the HR1 region of HIV gp41 and with one or more amino acid substitutions as described herein, which possess antiviral activity as evidenced by their ability to inhibit transmission of HIV to a target cell; and a method for inhibiting transmission of HIV to a target cell. Synthetic peptide according to the present invention, or trimers formed therefrom, may be used in the manufacture of a medicament or pharmaceutical composition for use in a method of inhibiting transmission of HIV-1 to a target cell. A method for inhibiting transmission of HIV-1 to a target cell comprises contacting the virus, in the presence of a target cell, with an amount of synthetic peptide according to the present invention (preferably in its self-assembled, trimeric form) effective to inhibit infection of the cell by HIV-1. Also, a method for inhibiting transmission of HIV-1 to a target cell comprises adding synthetic peptide according to the present invention (preferably as trimers) to the virus and the target cell in an amount effective to inhibit infection of the cell by HIV-1. Also provided is a method for inhibiting HIV (preferably, HIV-1) fusion (e.g., a process by which HIV gp41 mediates fusion between the viral membrane and cell membrane during infection by HIV of a target cell), comprising contacting the virus, in the presence of a target cell, with synthetic peptide according to the present invention (preferably in its self-assembled, trimeric form) in a concentration effective to inhibit HIV membrane fusion. These methods may be used to treat HIV-infected individuals (therapeutically) or to treat individuals newly exposed to or at high risk of exposure (e.g., through drug usage or high risk sexual behavior) to HIV (prophylactically). Thus, for example, in the case of an HIV-1 infected individual, an effective concentration (e.g., an effective amount) would be a dose sufficient (by itself and/or in conjunction with a regimen of doses) to reduce HIV-1 viral load in the individual being treated. As known to those skilled in the art, there are several standard methods for measuring HIV viral load which include, but are not limited to, by quantitative cultures of peripheral blood mononuclear cells and by plasma HIV RNA measurements. The synthetic peptides of the invention can be administered in a single administration, intermittently, periodically, or continuously, as can be determined by a medical practitioner, such as by monitoring viral load. Depending on the formulation containing synthetic peptide, and whether the synthetic peptide further comprises a macromolecular carrier, the synthetic peptides according to the present invention may be administered once or multiple times daily, periodically during a week period, or periodically during a month period. Further, the synthetic peptides according to the present invention may show synergistic results, of inhibiting transmission of HIV to a target cell, when used in combination (e.g., when used simultaneously, or in a cycling on with one drug and cycling off with another) with at least one other antiretroviral drug used for treatment of HIV (e.g., including, but not limited to, other HIV entry inhibitors (e.g., T20, T1249, and CCR5 inhibitors), HIV integrase inhibitors, reverse transcriptase inhibitors (e.g., nucleoside or nonnucleoside), protease inhibitors, and the like, as well known in the art). In this regard, synergistic results have been observed when T20 has been combined in therapy with at least one other antiretroviral drug (see, e.g., U.S. Pat. No. 6,475,491, the disclosure of which is herein incorporated by reference). The synergism observed is believed to be due, in part, to the mechanism of action of T20 and HIV fusion inhibitor peptides; i.e., T20 and the synthetic peptides (and trimers thereof) according to the present invention act by a different mechanism than classes of antiretroviral drugs other than HIV fusion inhibitor peptides.

Effective dosages of the synthetic peptides of the invention to be administered may be determined through procedures well known to those in the art; e.g., by determining potency, biological half-life, bioavailability, and toxicity. In a preferred embodiment, an effective synthetic peptide dosage range is determined by one skilled in the art using data from routine in vitro and in vivo studies well know to those skilled in the art. For example, in vitro infectivity assays of antiviral activity, such as described herein, enables one skilled in the art to determine the mean inhibitory concentration (IC) of the synthetic peptide necessary to block some amount of viral infectivity (e.g., 50% inhibition, IC₅₀; or 90% inhibition, IC₉₀). Appropriate doses can then be selected by one skilled in the art using pharmacokinetic data from one or more standard animal models, so that a minimum plasma concentration (C[min]) of the synthetic peptide is obtained which is equal to or exceeds a predetermined IC value. While dosage ranges typically depend on the route of administration chosen and the formulation of the dosage, an exemplary dosage range of the synthetic peptide according to the present invention may range from no less than 0.1 μg/kg body weight and no more than 10 mg/kg body weight; preferably a dosage range of from about 0.1-100 μg/kg body weight; and more preferably, a dosage of between from about 10 mg to about 250 mg of synthetic peptide. For example, if synthetic peptide according to the present invention further comprises a macromolecular carrier that causes synthetic peptide to remain active in the blood longer than synthetic peptide alone (i.e., in achieving a longer circulating plasma concentration), the amount of synthetic peptide in the dosage may be reduced as compared to the amount of synthetic peptide in a formulation not containing macromolecular carrier, and/or administered less frequently than a formulation not containing macromolecular carrier.

The compositions of the present invention (synthetic peptide, preferably self-assembled in solution into trimers) may be administered to an individual by any means that enables the active agent to reach the target cells (cells that can be infected by HIV). Thus, the compositions of this invention may be administered by any suitable technique, including oral, parenteral (e.g., intramuscular, intraperitoneal, intravenous, or subcutaneous injection or infusion, intradermal, or implant), nasal, pulmonary, vaginal, rectal, sublingual, or topical routes of administration, and can be formulated in dosage forms appropriate for each route of administration. The specific route of administration will depend, e.g., on the medical history of the individual, including any perceived or anticipated side effects from such administration, and the formulation of synthetic peptide being administered (e.g., the nature of the pharmaceutically acceptable carrier and/or macromolecular carrier of which synthetic peptide may further comprise). Most preferably, the administration is by injection (using, e.g., intravenous or subcutaneous means), but could also be by continuous infusion (using, e.g., slow-release devices or minipumps such as osmotic pumps, and the like). A formulation may comprise synthetic peptide according to the present invention which further comprises one or more of a pharmaceutically acceptable carrier and a macromolecular carrier; and may further depend on the site of delivery, the method of administration, the scheduling of administration, and other factors known to medical practitioners. A preferable formulation is one in which synthetic peptide according to the present invention is combined with or further comprises one or more of an agent, drug, reactive functionality, macromolecular carrier, or pharmaceutically acceptable carrier that inhibits or delays or retards the metabolism/degradation of synthetic peptide, particularly after it is administered to an individual. By way of example, injectable formulations, slow-release formulation, and oral formulations in which synthetic peptide (preferably self-assembled in trimers) of the invention is protected from hydrolysis by enzymes (e.g., digestive enzymes before absorption, proteolytic enzymes present in the blood, and the like) are embraced herein. Additionally, a formulation may comprise nucleotide sequences encoding synthetic peptide of the invention, as described herein in more detail, which upon administration, is expressed in cells of interest using techniques and expression vectors well known in the art.

### EXAMPLE 5

It is apparent to one skilled in the art, that based on the respective amino acid sequences of the synthetic peptides according to the present invention, that polynucleotides encoding such synthetic peptides may be synthesized or constructed, and that such synthetic peptides may be produced by recombinant DNA technology as a means of manufacture and/or (for example, in vivo production by introducing such polynucleotides in vivo as a means of gene or cell therapy) for a method of inhibiting transmission of HIV to a target cell. It is apparent to one skilled in the art that more than one polynucleotide sequence can encode a synthetic peptide according to the present invention, and that such polynucleotides may be synthesized on the basis of triplet codons known to encode the amino acids of the amino acid sequence of the synthetic peptide, third base degeneracy, and selection of triplet codon usage preferred by the host cell (e.g., prokaryotic or eukaryotic, species, etc,) in which expression is desired, For purposes of illustration only, and not limitation, examples of polynucleotides encoding synthetic peptide according to the present invention comprise SEQ ID NOs:47 to 51 (for synthetic peptides corresponding to 32, 35, 36, 29 and 30 respectively). However, it is understood that different codons can be substituted which code for the same amino acid(s) as the original codons. For example, SEQ ID NOs:47-51 encode the same respective synthetic peptides as SEQ ID NOs:52-56. However, SEQ ID NOs: 47-51 represent polynucleotides containing codon usage preferably for bacterial expression, whereas SEQ ID NOs:52-56 represent polynucleotides containing codon usage preferably for expression in mammalian expression systems. It is apparent to one skilled in the art from the codon usage as applied to these illustrated synthetic peptides may also be used to derive polynucleotides encoding other synthetic peptides of the present invention.

In one embodiment, provided is a prokaryotic expression vector containing a polynucleotide encoding a synthetic peptide according to the present invention, and its use for the recombinant production of synthetic peptide. In one example, the polynucleotide may be positioned in a prokaryotic expression vector so that when synthetic peptide is produced in bacterial host cells, it is produced as a fusion protein with sequences which assist in purification of the synthetic peptide. For example, there are sequences known to those skilled in the art which, as part of a fusion protein with a peptide desired to be expressed, facilitates production in inclusion bodies found in the cytoplasm of the prokaryotic cell used for expression (see. e.g., Tokatlidis et al., 1993, Protein Eng. 6:947-952). The inclusion bodies may be separated from other prokaryotic cellular components by methods known in the art to include denaturing agents, and fractionation (e.g., centrifugation, column chromatography, and the like). In another example, there are commercially available vectors into which is inserted a desired nucleic acid sequence of interest to be expressed as a protein or peptide such that upon expression, the gene product also contains a plurality of terminal histidine residues (“His tags”) that can be utilized in the purification of the gene product using methods standard in the art.

It is apparent to one skilled in the art that a nucleic acid sequence encoding a synthetic peptide according to the present invention can be inserted into a plasmid or vectors other than plasmids, and other expression systems can be used including, but not limited to, bacteria transformed with a bacteriophage vector, or cosmid DNA; yeast containing yeast vectors; fungi containing fungal vectors; insect cell lines infected with virus (e.g. baculovirus); and mammalian cell lines transfected with plasmid or viral expression vectors, or infected with recombinant virus (e.g. vaccinia virus, adenovirus, adeno-associated virus, retrovirus, etc.). Successful expression of the synthetic peptide requires that either the recombinant DNA molecule comprising the encoding sequence of the synthetic peptide, or the vector itself, contain the necessary control elements for transcription and translation which is compatible with, and recognized by the particular host system used for expression. Using methods known in the art of molecular biology, including methods described above, various promoters and enhancers can be incorporated into the vector or the recombinant DNA molecule comprising the encoding sequence to increase the expression of the synthetic peptide, provided that the increased expression of the synthetic peptide is compatible with (for example, non-toxic to) the particular host cell system used. As apparent to one skilled in the art, the selection of the promoter will depend on the expression system used. Promoters vary in strength, i.e., ability to facilitate transcription. Generally, for the purpose of expressing a cloned gene, it is desirable to use a strong promoter in order to obtain a high level of transcription of the gene and expression into gene product. For example, bacterial, phage, or plasmid promoters known in the art from which a high level of transcription has been observed in a host cell system comprising E. coli include the lac promoter, trp promoter, recA promoter, ribosomal RNA promoter, the P.sub.R and P.sub.L promoters, lacUV5, ompF, bla, lpp, and the like, may be used to provide transcription of the inserted nucleotide sequence encoding the synthetic peptide. Commonly used mammalian promoters in expression vectors for mammalian expression systems are the promoters from mammalian viral genes. Examples include the SV40 early promoter, mouse mammary tumor virus LTR promoter, adenovirus major late promoter, herpes simplex virus promoter, and the CMV promoter.

In the case where expression of the synthetic peptide may be lethal or detrimental to the host cells, the host cell strain/line and expression vectors may be chosen such that the action of the promoter is inhibited until specifically induced. For example, in certain operons the addition of specific inducers is necessary for efficient transcription of the inserted DNA (e.g., the lac operon is induced by the addition of lactose or isopropylthio-beta-D-galactoside; trp operon is induced when tryptophan is absent in the growth media; and tetracycline can be use in mammalian expression vectors having a tet sensitive promoter). Thus, expression of the synthetic peptide may be controlled by culturing transformed or transfected cells under conditions such that the promoter controlling the expression from the encoding sequence is not induced, and when the cells reach a suitable density in the growth medium, the promoter can be induced for expression from the encoding sequence. Other control elements for efficient gene transcription or message translation are well known in the art to include enhancers, transcription or translation initiation signals, transcription termination and polyadenylation sequences, and the like.

### EXAMPLE 6

In another preferred embodiment, synthetic peptide according to the present invention, or trimer formed therefrom, further comprises a macromolecular carrier. Such macromolecular carriers are well known in the art to include, but are not limited to, serum proteins, polymers, carbohydrates, lipids, fatty acids, and lipid-fatty acid conjugates. Serum proteins typically used as macromolecular carriers include, but are not limited to, transferrin (see e.g., Park et al., 1998, J. Drug Targeting, 6:53-64), albumin (Chuag et al., 2002, Pharm. Res. 19:569-577; J. Pharm. Exp. Therapeutics 2002, 303:540-548), immunoglobulins (preferably IgG or one or more chains thereof), or hormones; wherein the protein is preferably human, and more preferably a recombinant human protein. Polymers typically used as macromolecular carriers include, but are not limited to, polylysines or poly(D-L-alanine)-poly(L-lysine)s, or polyols. A preferred polyol comprises a water-soluble poly(alkylene oxide) polymer, and can have a linear or branched chain. Suitable polyols include, but are not limited to, polyethylene glycol (PEG), polypropylene glycol (PPG), and PEG-PPG copolymers.

In one example, the macromolecular carrier may be conjugated to synthetic peptide, or trimers formed therefrom. For example, in using a polyol, typically the polyol is derivatized or reacted with a coupling agent to form an “activated” polyol having one or more terminal reactive groups which can be used to react with a reactive functionality (e.g., preferably, a free amine group) of the synthetic peptide or trimers formed therefrom using methods standard in the art. Such reactive groups may include, but are not limited to, a hydroxy group, amino group, aldehyde group, and the like. The polyol used may comprise a linear chain or branched chain polymer. In another example, a synthetic peptide according to the present invention is synthesized, the last step of the synthesis process being the addition of a maleimide group (e.g., by a step in the solid phase synthesis of adding 3-maleimidoproprionic acid, washing, and then cleaving the synthetic peptide containing the maleimide group from the resin). Such methods are known in the art (see, e.g., WO 00/69902). The synthetic peptide, or trimers formed therefrom, may then be administered (preferably, parenterally) to an individual such that the synthetic peptide or trimers formed therefrom conjugate to a macromolecular carrier such as a blood component (preferably, a serum protein, and more preferably, albumin). In another example, recombinant human protein (e.g., albumin, transferrin, immunoglobulin, or the like) may be charged (“cationized”) and then thiolated using standard coupling agents known in the art (e.g., using N-succinimidyl S-acetylthioacetate). The thiolated, charged recombinant human protein may be coupled to avidin using standard coupling reagents known in the art (using m-maleimidobenzoyl-N-hydroxysuccinimide ester). The resultant avidinylated human protein may then be reacted with synthetic peptide, or trimers formed therefrom, which had been previously biotinylated using methods standard in the art. Thus, the result is synthetic peptide, or trimers formed therefrom, which have been linked to macromolecular carrier.

In an alternative example, the macromolecular carrier may be genetically expressed with synthetic peptide; e.g., as part of a fusion protein. For example, a DNA sequence encoding albumin may be cloned into a vector along with the DNA sequence encoding a linker and the DNA sequence encoding synthetic peptide according to the present invention, such that the resultant gene product is an albumin fusion protein comprising albumin with synthetic peptide linked at the C-terminal end, N-terminal end, or both the C-terminal and N-terminal ends of albumin. Such vectors and expression systems, preferably for yeast expression, are well known in the art (see, e.g., U.S. Pat. Nos. 5,728,553 & 5,965,386). Useful yeast plasmid vectors are generally commercially available (e.g., pRS403-406 series and pRS413-416 series) and which may incorporate the yeast selectable markers (e.g., his3, trp1, leu2, ura3, and the like).

An expression vector, containing a polynucleotide encoding an albumin-synthetic peptide fusion protein for yeast expression, may comprise an expression cassette comprising: a yeast promoter (e.g., a Sacchromyces PRB1 promoter); a sequence encoding a secretion leader which will facilitate secretion of the expressed gene product (e.g., could be the natural human albumin secretion leader and/or a yeast-derived secretion leader); a sequence encoding human albumin (e.g., as disclosed in Genbank); a sequence encoding a linker (e.g., the linker comprising a stretch of from about 4 to about 20 amino acids, and more preferably amino acids that include glycine and serine); a polynucleotide encoding synthetic peptide; and a transcription terminator (e.g., Saccharomyces ADH1). As apparent to one skilled in the art, if it is desired to have synthetic peptide being in the N-terminal region of the albumin fusion protein, then the polynucleotide encoding synthetic peptide would be placed between the promoter and the DNA encoding human albumin. The resultant expression vector may then be used to transform yeast, and culture conditions for recombinant production, as well as purification of the recombinant product, could be performed using methods known in the art. From these teachings and as apparent to one skilled in the art, similar constructs for producing synthetic peptide fused to a polypeptide other than albumin may be produced by using DNA encoding transferrin or DNA encoding IgG or encoding some other serum protein or macromolecular carrier, and modifying the expression vector and/or host cell for expression thereof. Thus, obtained can be synthetic peptide further comprising a macromolecular carrier (as a fusion protein), or a composition comprising synthetic peptide and macromolecular carrier, and may further comprise a pharmaceutically acceptable carrier.

In yet another example wherein the macromolecular carrier may be genetically expressed with synthetic peptide (e.g., as part of a fusion protein) DNA sequence encoding a molecule capable of binding the Fc-receptor FcRn may be cloned into a vector along with a DNA sequence encoding synthetic peptide according to the present invention (and may further comprise a DNA sequence encoding a linker), such that the resultant gene product is a fusion protein comprising synthetic peptide linked to the molecule capable of binding FcRn. Such molecules are known in the art to include whole IgG (preferably human IgG), the Fc Fragment of IgG, and other fragments of IgG that include the region(s) necessary for binding to FcRn. As disclosed in U.S. Pat. No. 6,030,613, it is known that the amino acids of the Fc region important for binding to FcRn include C.sub.H residues 248, 250-257, 272, 285, 288, 290-291, 308-311, 314, 385=387, 428, and 433-436. As apparent from the examples above, the synthetic peptide may be linked at the C-terminal end, N-terminal end, or both the C-terminal and N-terminal ends, of the molecule capable of binding FcRn. As apparent from the examples above, various expression systems known in the art may be used to produce such a composition comprising synthetic peptide and macromolecular carrier comprising molecule capable of binding FcRn. Such a composition may further include a pharmaceutically acceptable carrier to facilitate delivery as a pharmaceutical formulation.

### EXAMPLE 7

In another embodiment, the synthetic peptide according to the present invention (or a trimer formed therefrom) comprises an amino acid substitution comprising an addition of no less than 1 amino acid, and no more than about 20 amino acids to either or both of the amino terminus or carboxy terminus of the synthetic peptide, versus one or more amino acid substitutions in the amino acid sequence internal to both the amino terminus and carboxy terminus. Further, a preferred amino acid substitution may be used in the present invention to the exclusion of an amino acid substitution other than preferred amino acid substitution. This amino acid substitution may be used as the only modification (e.g., additional component) of the synthetic peptide (or trimer formed therefrom), or may be used in conjunction with one or more components comprising: (a) one or more reactive functionalities; (b) a pharmaceutically acceptable carrier; and (c) a macromolecular carrier.

For an example of this embodiment, U.S. Pat. Nos. 6,258,782, 6,348,568 and 6,562,787 disclose enhancer peptide sequences which may be linked to, at one or more of the carboxy terminus and amino terminus of, an antiviral HIV peptide. Such enhancer peptide sequences have been utilized to enhance the pharmacokinetic properties of an antiviral peptide to which it is linked. While the observed enhancement of pharmacokinetic properties is relative to the properties observed for the peptide to which it is linked, improvements in half-life (in vitro and/or in vivo in achieving a longer circulating plasma concentration) have been observed. Thus, in one embodiment of the synthetic peptide according to the present invention, the synthetic peptide comprises an amino acid substitution comprising an addition of an enhancer peptide sequence to either or both of the amino terminus or carboxy terminus of the synthetic peptide. The enhancer peptide sequences have the following consensus amino acid sequences: “WXXWXXXI” (SEQ ID NO:57); “WXXWXXX” (SEQ ID NO:58); “WXXWXX” (SEQ ID NO:59); “WXXWX” (SEQ ID NO:60); “WXXW” (SEQ ID NO:61); “WXXXWXWX” (SEQ ID NO:62); “XXXWXWX” (SEQ ID NO:63); “XXWXWX” (SEQ ID NO:64); “XWXWX” (SEQ ID NO:65); “WXWX” (SEQ ID NO:66); “WXXXWXW” (SEQ ID NO:67); “WXXXWX” (SEQ ID NO:68); “WXXXW” (SEQ ID NO:69); “IXXXWXXw” (SEQ ID NO:70); “XXXWXXW” (SEQ ID NO:71); “XXWXXW” (SEQ ID NO:72); “XWXXW” (SEQ ID NO:73); “XWXWXXXW” (SEQ ID NO:74); “XWXWXXX” (SEQ ID NO:75); “XWXWXX” (SEQ ID NO:76); “XWXW” (SEQ ID NO:77); “WXWXXXW” (SEQ ID NO:78); “XWXXXW” (SEQ ID NO:79); or “WXXXWXX” (SEQ ID NO:80); whereas X can be any amino acid, W represents trytophan, and I represents isoleucine. As discussed previously, the enhancer peptide sequences may also comprise peptide sequences that are otherwise the same as the consensus amino acid sequences but contain an amino acid substitution that does not abolish the ability of the peptide sequence to enhance the pharmacokinetic properties of the synthetic peptide to which it is linked relative to the pharmacokinetic properties of the synthetic peptide alone (not being linked to such enhancer peptide sequence), as determined by using standard methods known in the art for determining pharmacokinetic properties.

In another embodiment, the synthetic peptide according to the present invention comprises an amino acid substitution comprising an addition of a helix-capping motif to either or both of the amino terminus or carboxy terminus of the synthetic peptide. Helix-capping motifs are well known in the art to comprise no less than 1 amino acid and no more than about 20 amino acids that can be added (e.g., linked) to a peptide (or found in nature to be part of a polypeptide) in stabilizing the alpha helical structure of such peptide, particularly those containing leucine zipper motifs (see, e.g., Hollenbeck et al., 2002, Protein Sci 11:2740-7). As to effect on stability, and with respect to amino acid residues at the C terminal position, the rank order of amino acids has been reported to be as follows: Gly>His>Asn>Arg>Lys>Gln>Ala>Phe>Met>Ser>Asp>Glu>Trp>Thr>Pro>lle>Val (Thomas et al., 2001, Proc. Natl. Acad. Sci. USA 98:10670-5). Consensus helix capping motifs are known in the art (see, e.g., Aurora & Rose, 1998, Protein Sci. 7:21-38) to include, but are not limited to, (a) N-terminal capping motifs comprising “h-XpXhX”, “hp-XpXhX”, “hpp-XpXhX”, and (b) C-terminal capping motifs comprising “hXpX-Gh”, “hXpX-nXh”; “hXXX-GpXh”, and “hXXX-PpXh” (generally: where h is a hydrophobic amino acid, or neutral histidine; where p is a polar amino acid or protonated histidine; where n is not valine, isoleucine, threonine, or proline; where X is any amino acid; where G is glycine; and where P is proline). As apparent to one skilled in the art, a synthetic peptide according to the present invention may comprise amino acid substitution comprising an enhancer peptide linked at one terminus of the synthetic peptide, and a helix-capping motif linked to the terminus opposite that to which is linked the enhancer peptide.

The foregoing description of the specific embodiments of the present invention have been described in detail for purposes of illustration. In view of the descriptions and illustrations, others skilled in the art can, by applying current knowledge, readily modify and/or adapt the present invention for various applications without departing from the basic concept; and thus, such modifications and/or adaptations are intended to be within the meaning and scope of the appended claims.

What is claimed is:

1. A synthetic peptide comprising an amino acid sequence derived from the HR1 region of HIV-1 gp41; wherein the HR1 region consists of native amino acid sequence shown as SEQ ID NO:1 or polymorphisms thereof; wherein the HR1 region from which the synthetic peptide is derived comprises a hydrophobic domain of amino acids corresponding to amino acid residues in positions 28 to 36 of SEQ ID NO:1 or polymorphisms thereof; wherein the amino acid residues comprising the hydrophobic domain correspond to heptad repeat positions “efgabcdef”; and wherein the amino acid sequence of the synthetic peptide further comprises one or more amino acid substitutions in the heptad repeat positions “efgabcdef” comprising the hydrophobic domain, as compared to the native amino acid sequence of the HR1 region, which enables synthetic peptide to self-assemble in solution into trimers.

2. The synthetic peptide according to claim 1, wherein the one or more amino acid substitutions in the hydrophobic domain comprise either a substitution in the “c” position, or a substitution in both the “g” position and the “c” position, of the heptad repeat positions “efgabcdef”.

3. The synthetic peptide according to claim 2, wherein the synthetic peptide comprises an amino acid substitution additional to a substitution in either the “c” position or both the “g” position and “c” position, wherein the additional amino acid substitution is in one or more amino acid positions of one or more heptads of the synthetic peptide, and wherein the one or more amino acid positions is selected from the group consisting of an “a” position, a “d” position, a “b” position, and a combination thereof.

4. The synthetic peptide according to claim 1, wherein the one or more amino acid substitutions in the hydrophobic domain comprising the heptad repeat positions “efgabcdef” are in a position of the heptad repeat positions selected from the group consisting of a C-terminal “e” position, a C-terminal “f” position, and a combination thereof.

5. The synthetic peptide according to claim 4, wherein the synthetic peptide comprises an amino acid substitution additional to the substitution in one or more of the “e” position and the “f” position, wherein the additional amino acid substitution is in one or more amino acid positions of one or more heptads of the synthetic peptide, and wherein the one or more amino acid positions is selected from the group consisting of the “a” position, a “d” position, a “b” position, and a combination thereof.

6. The synthetic peptide according to claim 1, further comprising a component selected from the group consisting of one or more reactive functionalities, a pharmaceutically acceptable carrier, a macromolecular carrier, an amino acid substitution comprising an addition of no less than one amino acid and no more than twenty amino acids to either or both of the amino terminus or carboxy terminus of the synthetic peptide, and a combination thereof.

7. The synthetic peptide according to claim 2, further comprising a component selected from the group consisting of one or more reactive functionalities, a pharmaceutically acceptable carrier, a macromolecular carrier, an amino acid substitution comprising an addition of no less than one amino acid and no more than twenty amino acids to either or both of the amino terminus or carboxy terminus of the synthetic peptide, and a combination thereof.

8. The synthetic peptide according to claim 3, further comprising a component selected from the group consisting of one or more reactive functionalities, a pharmaceutically acceptable carrier, a macromolecular carrier, an amino acid substitution comprising an addition of no less than one amino acid and no more than twenty amino acids to either or both of the amino terminus or carboxy terminus of the synthetic peptide, and a combination thereof.

9. The synthetic peptide according to claim 4, further comprising a component selected from the group consisting of one or more reactive functionalities, a pharmaceutically acceptable carrier, a macromolecular carrier, an amino acid substitution comprising an addition of no less than one amino acid and no more than twenty amino acids to either or both of the amino terminus or carboxy terminus of the synthetic peptide, and a combination thereof.

10. The synthetic peptide according to claim 5, further comprising a component selected from the group consisting of one or more reactive functionalities, a pharmaceutically acceptable carrier, a macromolecular carrier, an amino acid substitution comprising an addition of no less than one amino acid and no more than twenty amino acids to either or both of the amino terminus or carboxy terminus of the synthetic peptide, and a combination thereof.

11. A trimer formed from synthetic peptide according to claim 1.

12. The trimer according to claim 11, further comprising a component selected from the group consisting of one or more reactive functionalities, a pharmaceutically acceptable carrier, a macromolecular carrier, an amino acid substitution comprising an addition of no less than one amino acid and no more than twenty amino acids to either or both of the amino terminus or carboxy terminus of synthetic peptide forming the trimer, and a combination thereof.

13. A trimer formed from synthetic peptide according to claim 2.

14. The trimer according to claim 13, further comprising a component selected from the group consisting of one or more reactive functionalities, a pharmaceutically acceptable carrier, a macromolecular carrier, an amino acid substitution comprising an addition of no less than one amino acid and no more than twenty amino acids to either or both of the amino terminus or carboxy terminus of synthetic peptide forming the trimer, and a combination thereof.

15. A trimer formed from synthetic peptide according to claim 3.

16. The trimer according to claim 15, further comprising a component selected from the group consisting of one or more reactive functionalities, a pharmaceutically acceptable carrier, a macromolecular carrier, an amino acid substitution comprising an addition of no less than one amino acid and no more than twenty amino acids to either or both of the amino terminus or carboxy terminus of synthetic peptide forming the trimer, and a combination thereof.

17. A trimer formed from synthetic peptide according to claim 4.

18. The trimer according to claim 17, further comprising a component selected from the group consisting of one or more reactive functionalities, a pharmaceutically acceptable carrier, a macromolecular carrier, an amino acid substitution comprising an addition of no less than one amino acid and no more than twenty amino acids to either or both of the amino terminus or carboxy terminus of synthetic peptide forming the trimer, and a combination thereof.

19. A trimer formed from synthetic peptide according to claim 5.

20. The trimer according to claim 19, further comprising a component selected from the group consisting of one or more reactive functionalities, a pharmaceutically acceptable carrier, a macromolecular carrier, an amino acid substitution comprising an addition of no less than one amino acid and no more than twenty amino acids to either or both of the amino terminus or carboxy terminus of synthetic peptide forming the trimer, and a combination thereof.

21. A synthetic peptide comprising an amino acid sequence derived from the HR1 region of HIV-1 gp41; wherein the amino acid sequence comprises a heptad repeat containing a plurality of heptads, and a hydrophobic domain comprising heptad repeat positions “efgabcdef” corresponding to amino acids 28 to 36 of SEQ ID NO:1 or polymorphisms thereof; wherein the synthetic peptide comprises an amino acid substitution in either the “c” position of the hydrophobic domain, or in both the “g” position and the “c” position of the hydrophobic domain, as compared to native sequence of the HR1 region; wherein the amino acid substitution enables the synthetic peptide to self-associate in solution into trimers.

22. The synthetic peptide according to claim 21, wherein the synthetic peptide comprises an amino acid substitution, as compared to native sequence of the HR1 region, additional to a substitution in a “c” position or in both the “g” position and “c” position; wherein the additional amino acid substitution is in one or more heptads of the synthetic peptide; and wherein the additional amino acid substitution is in one or more amino acid positions selected from the group consisting of an “a” position, a “d” position, a “b” position, and a combination thereof.

23. The synthetic peptide according to claim 21, further comprising a component selected from the group consisting of one or more reactive functionalities, a pharmaceutically acceptable carrier, a macromolecular carrier, an amino acid substitution comprising an addition of no less than one amino acid and no more than twenty amino acids to either or both of the amino terminus or carboxy terminus of the synthetic peptide, and a combination thereof.

24. The synthetic peptide according to claim 22, further comprising a component selected from the group consisting of one or more reactive functionalities, a pharmaceutically acceptable carrier, a macromolecular carrier, an amino acid substitution comprising an addition of no less than one amino acid and no more than twenty amino acids to either or both of the amino terminus or carboxy terminus of the synthetic peptide, and a combination thereof.

25. A trimer formed from synthetic peptide according to claim 21.

26. The trimer according to claim 25, further comprising a component selected from the group consisting of one or more reactive functionalities, a pharmaceutically acceptable carrier, a macromolecular carrier, an amino acid substitution comprising an addition of no less than one amino acid and no more than twenty amino acids to either or both of the amino terminus or carboxy terminus of synthetic peptide forming the trimer, and a combination thereof.

27. A trimer formed from synthetic peptide according to claim 22.

28. The trimer according to claim 27, further comprising a component selected from the group consisting of one or more reactive functionalities, a pharmaceutically acceptable carrier, a macromolecular carrier, an amino acid substitution comprising an addition of no less than one amino acid and no more than twenty amino acids to either or both of the amino terminus or carboxy terminus of synthetic peptide forming the trimer, and a combination thereof.

29. A synthetic peptide comprising an amino acid sequence derived from the HR1 region of HIV-1 gp41; wherein the amino acid sequence comprises a heptad repeat containing a plurality of heptads, and a hydrophobic domain comprising heptad repeat positions “efgabcdef” corresponding to amino acids 28 to 36 of SEQ ID NO:1 or polymorphisms thereof; wherein the synthetic peptide comprises an amino acid substitution in one or more of an “e” position at the C-terminus of the hydrophobic domain, an “f” position at the C-terminus of the hydrophobic domain, or a combination thereof, as compared to native sequence of the HR1 region; and wherein the amino acid substitution enables the synthetic peptide to self-associate in solution into trimers.

30. The synthetic peptide according to claim 29, wherein the synthetic peptide comprises an amino acid substitution, as compared to native sequence of the HR1 region, additional to the substitution in one or more of an “e” position and “f” position; wherein the additional amino acid substitution is in one or more heptads of the synthetic peptide; and wherein the additional amino acid substitution is in one or more amino acid positions selected from the group consisting of an “a” position, a “d” position, a “b” position, and a combination thereof.

31. The synthetic peptide according to claim 29, further comprising a component selected from the group consisting of one or more reactive functionalities, a pharmaceutically acceptable carrier, a macromolecular carrier, an amino acid substitution comprising an addition of no less than one amino acid and no more than twenty amino acids to either or both of the amino terminus or carboxy terminus of the synthetic peptide, and a combination thereof.

32. The synthetic peptide according to claim 30, further comprising a component selected from the group consisting of one or more reactive functionalities, a pharmaceutically acceptable carrier, a macromolecular carrier, an amino acid substitution comprising an addition of no less than one amino acid and no more than twenty amino acids to either or both of the amino terminus or carboxy terminus of the synthetic peptide, and a combination thereof.

33. A trimer formed from synthetic peptide according to claim 29.

34. The trimer according to claim 33, further comprising a component selected from the group consisting of one or more reactive functionalities, a pharmaceutically acceptable carrier, a macromolecular carrier, an amino acid substitution comprising an addition of no less than one amino acid and no more than twenty amino acids to either or both of the amino terminus or carboxy terminus of synthetic peptide forming the trimer, and a combination thereof.

35. A trimer formed from synthetic peptide according to claim 30.

36. The trimer according to claim 35, further comprising a component selected from the group consisting of one or more reactive functionalities, a pharmaceutically acceptable carrier, a macromolecular carrier, an amino acid substitution comprising an addition of no less than one amino acid and no more than twenty amino acids to either or both of the amino terminus or carboxy terminus of synthetic peptide forming the trimer, and a combination thereof.

37. A synthetic peptide comprising an amino acid sequence selected from the group of amino acid sequences consisting of: SEQ ID NO:29, SEQ ID NO:30, SEQ ID NO:31, SEQ ID NO:32, SEQ ID NO:34, SEQ ID NO:35, SEQ ID NO:36, SEQ ID NO:37, SEQ ID NO:38, SEQ ID NO:39, SEQ ID NO:40, SEQ ID NO:43, SEQ ID NO:81, and SEQ ID NO:82.

38. The synthetic peptide according to claim 37, further comprising a component selected from the group consisting of one or more reactive functionalities, a pharmaceutically acceptable carrier, a macromolecular carrier, an amino acid substitution comprising an addition of no less than one amino acid and no more than twenty amino acids to either or both of the amino terminus or carboxy terminus of the synthetic peptide, and a combination thereof.

39. A trimer formed from synthetic peptide according to claim 37.

40. The trimer according to claim 39, further comprising a component selected from the group consisting of one or more reactive functionalities, a pharmaceutically acceptable carrier, a macromolecular carrier, an amino acid substitution comprising an addition of no less than one amino acid and no more than twenty amino acids to either or both of the amino terminus or carboxy terminus of synthetic peptide forming the trimer, and a combination thereof.

41. A trimer formed from self-association of synthetic peptide in solution, wherein the synthetic peptide comprises an amino acid sequence derived from the HR1 region of HIV-1 gp41; wherein the HR1 region consists of native amino acid sequence shown as SEQ ID NO:1 or polymorphisms thereof; wherein the HR1 region from which the synthetic peptide is derived comprises a hydrophobic domain of amino acids corresponding to amino acid residues 28 to 36 of SEQ ID NO:1 or polymorphisms thereof; wherein the amino acid residues comprising the hydrophobic domain correspond to heptad repeat positions “efgabcdef”; and wherein the amino acid sequence of the synthetic peptide further comprises one or more amino acid substitutions in the heptad repeat positions “efgabcdef” comprising the hydrophobic domain, as compared to native amino acid sequence of the HR1 region, which enables synthetic peptide to self-associate in solution into trimers.

42. The trimer according to claim 39, further comprising a component selected from the group consisting of one or more reactive functionalities, a pharmaceutically acceptable carrier, a macromolecular carrier, an amino acid substitution comprising an addition of no less than one amino acid and no more than twenty amino acids to either or both of the amino terminus or carboxy terminus of synthetic peptide forming the trimer, and a combination thereof.

43. A trimer formed from self-association of synthetic peptide in solution, wherein the synthetic peptide comprises an amino acid sequence derived from the HR1 region of HIV-1 gp41; wherein the amino acid sequence comprises a heptad repeat containing a plurality of heptads, and a hydrophobic domain comprising heptad repeat positions “efgabcdef” corresponding to amino acids 28 to 36 of SEQ ID NO:1 or polymorphisms thereof; wherein the synthetic peptide comprises an amino acid substitution in either the “c” position of the hydrophobic domain, or in both the “g” position and the “c” position of the hydrophobic domain, as compared to native sequence of the HR1 region; and wherein the amino acid substitution enables the synthetic peptide to self-associate in solution into trimers.

44. The trimer according to claim 43, further comprising a component selected from the group consisting of one or more reactive functionalities, a pharmaceutically acceptable carrier, a macromolecular carrier, an amino acid substitution comprising an addition of no less than one amino acid and no more than twenty amino acids to either or both of the amino terminus or carboxy terminus of synthetic peptide forming the trimer, and a combination thereof.

45. A trimer formed from self-association of synthetic peptide in solution, wherein the synthetic peptide comprises an amino acid sequence derived from the HR1 region of HIV-1 gp41; wherein the amino acid sequence comprises a heptad repeat containing a plurality of heptads, and a hydrophobic domain comprising heptad repeat positions “efgabcdef” corresponding to amino acids 28 to 36 of SEQ ID NO:1 or polymorphisms thereof; wherein the synthetic peptide comprises an amino acid substitution in either the “c” position of the hydrophobic domain or in both the “g” position and the “c” position of the hydrophobic domain, as compared to native sequence of the HR1 region; wherein the synthetic peptide also comprises an amino acid substitution, additional to the substitution in the “c” position or in both the “g” position and “c” position, in one or more heptads of the synthetic peptide; wherein the additional amino acid substitution is in one or more amino acid positions selected from the group consisting of an “a” position, a “d” position, a “b” position, and a combination thereof; and wherein the amino acid substitutions enable the synthetic peptide to self-associate in solution into trimers.

46. The trimer according to claim 45, further comprising a component selected from the group consisting of one or more reactive functionalities, a pharmaceutically acceptable carrier, a macromolecular carrier, an amino acid substitution comprising an addition of no less than one amino acid and no more than twenty amino acids to either or both of the amino terminus or carboxy terminus of synthetic peptide forming the trimer, and a combination thereof.

47. A trimer formed from self-association of synthetic peptide in solution, wherein the synthetic peptide comprises an amino acid sequence derived from the HR1 region of HIV-1 gp41; wherein the amino acid sequence comprises a heptad repeat containing a plurality of heptads, and a hydrophobic domain comprising heptad repeat positions “efgabcdef” corresponding to amino acids 28 to 36 of SEQ ID NO:1 or polymorphisms thereof; wherein the synthetic peptide comprises an amino acid substitution in one or more of an “e” position at the C-terminus of the hydrophobic domain, an “f” position at the C-terminus of the hydrophobic domain, or a combination thereof, as compared to native sequence of the HR1 region; and wherein the amino acid substitution enables the synthetic peptide to self-associate in solution into trimers.

48. The trimer according to claim 47, further comprising a component selected from the group consisting of one or more reactive functionalities, a pharmaceutically acceptable carrier, a macromolecular carrier, an amino acid substitution comprising an addition of no less than one amino acid and no more than twenty amino acids to either or both of the amino terminus or carboxy terminus of synthetic peptide forming the trimer, and a combination thereof.

49. A trimer formed from self-association of synthetic peptide in solution, wherein the synthetic peptide comprises an amino acid sequence derived from the HR1 region of HIV-1 gp41; wherein the amino acid sequence comprises a heptad repeat containing a plurality of heptads, and a hydrophobic domain comprising heptad repeat positions “efgabcdef” corresponding to amino acids 28 to 36 of SEQ ID NO:1 or polymorphisms thereof; wherein the synthetic peptide comprises an amino acid substitution in one or more of an “e” position at the C-terminus of the hydrophobic domain, an “f” position at the C-terminus of the hydrophobic domain, or a combination thereof, as compared to the native sequence of the HR1 region; wherein the synthetic peptide also comprises an amino acid substitution, additional to the substitution in either or both of the “e” position and the “f” position, in one or more heptads of the synthetic peptide; wherein the additional amino acid substitution is in one or more amino acid positions selected from the group consisting of an “a” position, a “d” position, a “b” position”, and a combination thereof; and wherein the amino acid substitutions enable the synthetic peptide to self-associate in solution into trimers.

50. The trimer according to claim 49, further comprising a component selected from the group consisting of one or more reactive functionalities, a pharmaceutically acceptable carrier, a macromolecular carrier, an amino acid substitution comprising an addition of no less than one amino acid and no more than twenty amino acids to either or both of the amino terminus or carboxy terminus of synthetic peptide forming the trimer, and a combination thereof.

51. A trimer formed from self association of synthetic peptide in solution, wherein the synthetic peptide comprises an amino acid sequence selected from the group of amino acid sequences consisting of: SEQ ID NO:29, SEQ ID NO:30, SEQ ID NO:31, SEQ ID NO:32, SEQ ID NO:34, SEQ ID NO:35, SEQ ID NO:36, SEQ ID NO:37, SEQ ID NO:38, SEQ ID NO:39, SEQ ID NO:40, SEQ ID NO:43, SEQ ID NO:81, and SEQ ID NO:82.

52. The trimer according to claim 49, further comprising a component selected from the group consisting of one or more reactive functionalities, a pharmaceutically acceptable carrier, a macromolecular carrier, an amino acid substitution comprising an addition of no less than one amino acid and no more than twenty amino acids to either or both of the amino terminus or carboxy terminus of synthetic peptide forming the trimer, and a combination thereof.

53. A method for inhibition of transmission of HIV-1 to a cell comprising contacting the virus, in the presence of a target cell, with synthetic peptide in a concentration effective to inhibit infection of the cell by HIV-1, thereby inhibiting transmission of HIV-1 to the cell, wherein:
the synthetic peptide comprises an amino acid sequence derived from the HR1 region of HIV-1 gp41;
the HR1 region consists of native amino acid sequence shown as SEQ ID NO:1 or polymorphisms thereof;
the HR1 region from which the synthetic peptide is derived comprises a hydrophobic domain of amino acids comprising the sequence corresponding to amino acid residues 28 to 36 of SEQ ID NO:1 or polymorphisms thereof;
amino acid residues comprising the hydrophobic domain correspond to heptad repeat positions “efgabcdef”; and
the amino acid sequence of the synthetic peptide comprises one or more amino acid substitutions in the heptad repeat positions “efgabcdef” comprising the hydrophobic domain, as compared to the native amino acid sequence of the HR1 region, which enables synthetic peptide to self-assemble in solution into trimers.

54. The method according to claim 53, wherein the one or more amino acid substitutions in the hydrophobic domain comprise a substitution in either the “c” position or in both the “g” position and the “c” position of the heptad repeat positions “efgabcdef”.

55. The method according to claim 54, wherein the synthetic peptide comprises an amino acid substitution additional to the substitution in either the “c” position or in both the “g” position and “c” position, wherein the additional amino acid substitution is in one or more amino acid positions of one or more heptads of the synthetic peptide, and wherein the one or more amino acid positions is selected from the group consisting of an “a” position, a “d” position, a “b” position, and a combination thereof.

56. The method according to claim 53, wherein the one or more amino acid substitutions in the hydrophobic domain comprising the heptad repeat positions “efgabcdef” are selected from the group consisting of a C-terminal “e” position, a C-terminal “f” position, and a combination thereof.

57. The method according to claim 56, wherein the synthetic peptide comprises an amino acid substitution additional to the substitution in one or more of the “e” position and the “” position, wherein the additional amino acid substitution is in one or more amino acid positions of one or more heptads of the synthetic peptide, and wherein the one or more amino acid positions is selected from the group consisting of an “a” position, a “d” position, a “b” position, and a combination thereof.

58. The method according to claim 53, wherein synthetic peptide further comprises a component selected from the group consisting of one or more reactive functionalities, a pharmaceutically acceptable carrier, a macromolecular carrier, an amino acid substitution comprising an addition of no less than one amino acid and no more than twenty amino acids to either or both of the amino terminus or carboxy terminus of the synthetic peptide, and a combination thereof.

59. The method according to claim 53, wherein synthetic peptide is in an oligomeric form comprising trimers.

60. The method according to claim 59, wherein the trimers further comprise a component selected from the group consisting of one or more reactive functionalities, a pharmaceutically acceptable carrier, a macromolecular carrier, an amino acid substitution comprising an addition of no less than one amino acid and no more than twenty amino acids to either or both of the amino terminus or carboxy terminus of synthetic peptide forming the trimers, and a combination thereof.

61. The method of claim 53, wherein the synthetic peptide is parenterally administered to an individual.

62. A method for inhibition of transmission of HIV-1 to a target cell comprising adding synthetic peptide to the virus and a target cell in an amount effective to inhibit infection of the cell by HIV-1, wherein:
the synthetic peptide comprises an amino acid sequence derived from the HR1 region of HIV-1 gp41;
the HR1 region consists of native amino acid sequence shown as SEQ ID NO:1 or polymorphisms thereof;
the HR1 region from which the synthetic peptide is derived comprises a hydrophobic domain of amino acids comprising the sequence corresponding to amino acid residues 28 to 36 of SEQ ID NO:1 or polymorphisms thereof;
amino acid residues comprising the hydrophobic domain correspond to heptad repeat positions “efgabcdef”; and
the amino acid sequence of the synthetic peptide comprises one or more amino acid substitutions in the heptad repeat positions “efgabcdef” comprising the hydrophobic domain, as compared to the native amino acid sequence of the HR1 region, which enables synthetic peptide to self-assemble in solution into trimers.

63. The method according to claim 62, wherein the one or more amino acid substitutions in the hydrophobic domain comprise a substitution in either the “c” position, or in both the “g” position and the “c” position, of the heptad repeat positions “efgabcdef”.

64. The method according to claim 63, wherein the synthetic peptide comprises an amino acid substitution additional to a substitution in either the “c” position or both the “g” position and “c” position, wherein the additional amino acid substitution is in one or more amino acid positions of one or more heptads of the synthetic peptide, and wherein the one or more amino acid positions is selected from the group consisting of an “a” position, a “d” position, a “b” position, and a combination thereof.

65. The method according to claim 62, wherein the one or more amino acid substitutions in the hydrophobic domain comprising the heptad repeat positions “efgabcdef” are selected from the group consisting of a C-terminal “e” position, a C-terminal “f” position, and a combination thereof.

66. The method according to claim 65, wherein the synthetic peptide comprises an amino acid substitution additional to the substitution in one or more of the “e” position and the “f” position, wherein the additional amino acid substitution is in one or more amino acid positions of one or more heptads of the synthetic peptide, and wherein the one or more amino acid positions is selected from the group consisting of an “a” position, a “d” position, a “b” position, and a combination thereof.

67. The method according to claim 62, wherein synthetic peptide further comprises a component selected from the group consisting of one or more reactive functionalities, a pharmaceutically acceptable carrier, a macromolecular carrier, an amino acid substitution comprising an addition of no less than one amino acid and no more than twenty amino acids to either or both of the amino terminus or carboxy terminus of the synthetic peptide, and a combination thereof.

68. The method according to claim 62, wherein synthetic peptide is in an oligomeric form comprising trimers.

69. The method according to claim 68, wherein the trimers further comprise a component selected from the group consisting of one or more reactive functionalities, a pharmaceutically acceptable carrier, a macromolecular carrier, an amino acid substitution comprising an addition of no less than one amino acid and no more than twenty amino acids to either or both of the amino terminus or carboxy terminus of synthetic peptide forming the trimers, and a combination thereof.

70. The method of claim 62, wherein synthetic peptide is parenterally administered to an individual.

71. A method for inhibiting HIV fusion with a target cell comprising contacting the virus, in the presence of a target cell, with synthetic peptide in a concentration effective to inhibit membrane fusion between the virus and the cell, wherein:
the synthetic peptide comprises an amino acid sequence derived from the HR1 region of HIV-1 gp41;
the HR1 region consists of native amino acid sequence shown as SEQ ID NO:1 or polymorphisms thereof;
the HR1 region from which the synthetic peptide is derived comprises a hydrophobic domain of amino acids comprising the sequence corresponding to amino acid residues 28 to 36 of SEQ ID NO:1 or polymorphisms thereof;
amino acid residues comprising the hydrophobic domain correspond to heptad repeat positions “efgabcdef”; and
the amino acid sequence of the synthetic peptide comprises one or more amino acid substitutions in the heptad repeat positions “efgabcdef” comprising the hydrophobic domain, as compared to the native amino acid sequence of the HR1 region, which enables synthetic peptide to self-assemble in solution into trimers.

72. The method according to claim 71, wherein the one or more amino acid substitutions in the hydrophobic domain comprise a substitution in either the “c” position or in both the “g” position and the “c” position of the heptad repeat positions “efgabcdef”.

73. The method according to claim 72, wherein the synthetic peptide comprises an amino acid substitution additional to the substitution in the “c” position or in both the “g” position and “c” position, wherein the additional amino acid substitution is in one or more amino acid positions of one or more heptads of the synthetic peptide, and wherein the one or more amino acid positions is selected from the group consisting of the “a” position, a “d” position, a “b” position, and a combination thereof.

74. The method according to claim 71, wherein the one or more amino acid substitutions in the hydrophobic domain comprising the heptad repeat positions “efgabcdef” are selected from the group consisting of a C-terminal “e” position, a C-terminal “f” position, and a combination thereof.

75. The method according to claim 74, wherein the synthetic peptide comprises an amino acid substitution additional to the substitution in one or more of the “e” position and the “f” position, wherein the additional amino acid substitution is in one or more amino acid positions of one or more heptads of the synthetic peptide, and wherein the one or more amino acid positions is selected from the group consisting of an “a” position, a “d” position, a “b” position, and a combination thereof.

76. The method according to claim 71, wherein synthetic peptide further comprises a component selected from the group consisting of one or more reactive functionalities, a pharmaceutically acceptable carrier, a macromolecular carrier, an amino acid substitution comprising an addition of no less than one amino acid and no more than twenty amino acids to either or both of the amino terminus or carboxy terminus of the synthetic peptide, and a combination thereof.

77. The method according to claim 71, wherein synthetic peptide is in an oligomeric form comprising trimers.

78. The method according to claim 77, wherein the trimers further comprise a component selected from the group consisting of one or more reactive functionalities, a pharmaceutically acceptable carrier, a macromolecular carrier, an amino acid substitution comprising an addition of no less than one amino acid and no more than twenty amino acids to either or both of the amino terminus or carboxy terminus of synthetic peptide forming the trimers, and a combination thereof.

79. The method of claim 71, wherein synthetic peptide is parenterally administered to an individual.
